(19) United States
(12) Patent Application Publication
(10) Pub. No.: US 2025/0051459 A1
(43) Pub. Date: Feb. 13, 2025

(54) WNT-SURROGATE AGENTS AND METHODS FOR LACRIMAL GLAND REGENERATION

(71) Applicant: Surrozen Operating, Inc., South San Francisco, CA (US)

(72) Inventors: Yang LI, Mountain View, CA (US); Huy Tuan NGUYEN, Palo Alto, CA (US); Yorick POST, Palo Alto, CA (US); Wen-Chen YEH, Belmont, CA (US)

(21) Appl. No.: 18/719,798

(22) PCT Filed: Dec. 16, 2022
(86) PCT No.: PCT/US2022/081884

§ 371 Date: Jun. 13, 2024

Related U.S. Application Data
(60) Provisional application No. 63/291,243, filed on Dec. 17, 2021.

(51) Int. Cl. C07K 16/28; A61K 38/00; A61K 39/00; A61P 27/02; C07K 14/475

(52) U.S. Cl. CPC C07K 16/2863; A61P 27/02; C07K 14/475; A61K 38/00; A61K 2039/505; C07K 2317/31

(57) ABSTRACT

Methods and compositions for treatment of dry-eye disorders are provided. In particular, the present disclosure provides methods and compositions for modulating WNT (Wingless and lnt-1) signaling in a subject to modulate regeneration of tear-producing acinar cells to treat aqueous-deficient type dry-eye diseases. Further disclosed are WNT signaling modulators used in the methods.

### CROSS REFERENCE TO RELATED APPLICATIONS

This application claims priority to U.S. Provisional Application No. 63/291,243 filed on Dec. 17, 2021, each of which is incorporated by reference herein in its entirety.

### STATEMENT REGARDING SEQUENCE LISTING

The Sequence Listing XML associated with this application is provided in XML file format and is hereby incorporated by reference into the specification. The name of the XML file containing the Sequence Listing XML is SRZN_024_01WO_ST26.xml. The XML file is 61,424 bytes, was created on Dec. 15, 2022, and is being submitted electronically via USPTO Patent Center.

### FIELD OF INVENTION

The present disclosure relates to WNT signal modulators and treatment methods for various ophthalmology associated disorders.

### BACKGROUND OF THE INVENTION

The lacrimal gland is an exocrine gland positioned under the outer lateral portion of the upper eyelid in most vertebrates. The lacrimal gland produces a tear film, which is secreted by lacrimal ducts, and is associated with ocular health. (See Yao & Zhang at 939.) The film keeps the cornea surface and inner eyelids moistened and protects the corneal and conjunctival epithelia from physical damage, as well as from immune reactions. (See id.)

Lacrimal gland disorders and damage can cause significant ocular disease pathologies, especially dry-eye disease. (Dartt, OCULAR DISEASE at 105) Dry-eye disease is characterized by increased osmolarity of the tear film, and inflammation of the ocular surface. (See id.) Inadequate production of tears can lead to irritation, pain, and potentially damage to the ocular surface.

There are two major classes of dry-eye diseases: aqueous-deficient and evaporative. In turn, aqueous deficiency is subclassed into Sjögren's syndrome (autoimmune) dry-eye and non-Sjögren's syndrome (non-autoimmune) dry-eye.

In cases of aqueous-deficient dry-eye, the lacrimal gland is frequently inflamed, leading to atrophy and cell death of the tear-fluid-producing acinar cells. Following loss of acinar cells, tear production problems worsen leading to a reinforcing vicious inflammation-atrophy cycle. Accordingly, the lacrimal gland is a target in aqueous-deficient dry-eye diseases.

Presently, both aqueous-deficient and evaporative dry-eye diseases are treated by topical administration on the ocular surface of artificial tears and/or anti-inflammatory agents. (See id. at 106.) Only a few treatments target the lacrimal gland itself, by systemic administration of anti-inflammatory agents, so-called secretagogue agents, immunosuppressive agents, and sex hormones. (See id.) None of these treatment approaches deal directly with atrophy and/or damage to the lacrimal gland, including the tear-producing acinar cells. Consequently, there is a great unmet need for agents and methods for regeneration of acinar cells, restoring endogenous tear production and breaking the cycle of inflammation and atrophy.

### BRIEF SUMMARY

The present description provides methods of treating dry-eye disorders by activation and/or regeneration of lacrimal gland and/or lacrimal gland acinar cells, and compositions therefor.

In an aspect, the present invention comprises a method of regenerating lacrimal gland acinar cells, progenitor cells, ductal cells, myoepithelial cells, or immune cells in a subject, comprising administering a WNT signaling modulator to the subject. In an embodiment, the WNT signaling modulator may be an engineered WNT signaling modulator. In another embodiment, the WNT signaling modulator is an engineered WNT agonist or superagonist, or an engineered WNT antagonist. In particular embodiments, the cells are epithelial stem and/or progenitor cells, e.g., lacrimal gland epithelial stem and/or progenitor cells.

In an embodiment of the method of regenerating lacrimal gland acinar cells, the WNT signaling modulator may comprise at least one engineered bi-specific full-length IgG antibody that directly activates a canonical WNT signaling pathway. In any embodiment, the engineered WNT agonist may be selected from: (i) WNT3a; (ii) a WNT mimetic; or (iii) an R-spondin mimetic. The WNT mimetic may be a SWAP™ compound. The R-spondin mimetic may be a SWEETS™ compound.

In an embodiment of the method of regenerating lacrimal gland acinar cells, the WNT signaling modulator may affect expression of any one or more of: Fzd1, Fzd2, Fzd5, Fzd7, Fzd8, and Lrp6, and/or Lrp5. For example, the WNT signaling modulator may target any one or more of the group consisting of: Fzd1, Fzd2, and Fzd7; or any one or more of the group consisting of: Fzd5 and Fzd8, while also targeting Lrp6 and/or Lrp5. For another example, the WNT signaling modulator may affect expression of any one or more of: Fzd1, Fzd2, and Fzd7.

In one embodiment of the method of regenerating lacrimal gland acinar cells, the method further comprises the step of administering at least one of the group consisting of: RSPO2, RSPO2 fragment, and engineered RSPO2 mimetic.

In any embodiment, the WNT signaling modulator may be in a concentration ≥1 nM. In any embodiment, the WNT signaling modulator may be administered in a therapeutically effective amount. In any embodiment, the subject may be a live mammal. In any embodiment, the subject may be a human patient.

In another aspect, the present invention comprises a method of treating a lacrimal gland disorder in a subject, comprising administering a WNT signaling modulator to the subject. In an embodiment, the WNT signaling modulator may be an engineered WNT signaling modulator. In another embodiment, the WNT signaling modulator is an engineered WNT agonist or an engineered WNT antagonist. In further embodiment, the WNT signaling modulator is an engineered WNT superagonist.

In an embodiment of the method of treating a lacrimal gland disorder, the WNT signaling modulator may comprise at least one engineered bi-specific full-length IgG antibody that directly activates a canonical WNT signaling pathway. In any embodiment, the engineered WNT agonist may be selected from: (i) WNT3a; (ii) a WNT mimetic; or (iii) an R-spondin mimetic. The WNT mimetic may be a SWAP™ compound. The R-spondin mimetic may be a SWEETS™ compound.

In an embodiment of the method of treating a lacrimal gland disorder, the WNT signaling modulator may affect expression of any one or more of: Fzd1, Fzd2, Fzd5, Fzd7, Fzd8, and Lrp6, and/or Lrp5. For example, the WNT signaling modulator may target any one or more of the group consisting of: Fzd1, Fzd2, and Fzd7; or any one or more of the group consisting of: Fzd5 and Fzd8, while also targeting Lrp6 and/or Lrp5. For another example, the WNT signaling modulator may affect expression of any one or more of: Fzd1, Fzd2, and Fzd7.

In one embodiment of the method of treating a lacrimal gland disorder, the method further comprises the step of administering at least one of the group consisting of: RSPO2, RSPO2 fragment, and engineered RSPO2 mimetic.

In any embodiment of the method of treating a lacrimal gland disorder, the WNT signaling modulator may be in a concentration ≥1 nM. In any embodiment, the WNT signaling modulator may be administered in a therapeutically effective amount. In any embodiment, the subject may be a live mammal. In any embodiment, the subject may be a human patient.

In still another aspect, the present invention comprises a composition for the treatment of a dry-eye disorder in a subject, the composition comprising a WNT signaling modulator. In any embodiment of the composition, the dry-eye disorder may be due to Sjögren's syndrome disorder, chronic graft versus host disease (cGHVD), rheumatoid arthritis (RA), Stephen's Johnson syndrome, Ocular Rosacea, chemotherapy, radiation oncology treatments, diabetes, lupus, etc.

In an embodiment of the composition, the WNT signaling modulator may comprise at least one engineered bi-specific full-length IgG antibody that directly activates a canonical WNT signaling pathway. The at least one engineered bi-specific full-length IgG antibody may be specific for any one or more of Fzd1, Fzd,2, Fzd5, Fzd7, Fzd8, and Lrp6, and or Lrp5.

In an embodiment, the composition may further comprise an anti-inflammatory agent or lacrimal gland secretagogue.

In any embodiment of the composition, the composition may comprise therapeutically effective amounts of each of its components.

In any embodiment of the composition, the subject may be a live mammal. In any embodiment of the composition, the subject may be a human patient.

### BRIEF DESCRIPTION OF THE DRAWINGS

FIG. 1 depicts representative optical micrograph images of organoid outgrowth from primary mouse lacrimal gland tissue after 7 days using a standard 3D organoid protocol in Advanced DMEM supplemented with additional growth factors. Conditions include Base containing R-spondin 1 (RSPO1) alone (control, upper left), or the addition of various WNT mimetic compounds, each at 5 nM concentration. “F” represents various FZD binders (e.g., 18R5—a FZD 1,2,5,7,8 binder (“F12578”); R2H1—a FZD1,2,7 binder (“F127”); 2919—a FZD 5,8 binder (“F58”); 5063—a FZD4 binder (“F4”); and HB9L9.3—a FZD10 binder (F10)) that bind to various FZD receptors; L is an LRP binder, YW211.31.57, that binds to LRP5 and LRP6.

FIG. 2 depicts a micrograph image showing lacrimal organoid morphology (left), fluorescence indicating Mist1 acinar cell marker gene expression (center) and composite (right), scale bar is 100 μm.

FIGS. 3A-3C depict bar graphs indicating relative gene expression of either proliferative markers or markers specific for lacrimal-derived cells and acinar cells, in profiled organoids expanded for 7 days in control medium or in medium with L6-F12578 WNT mimetic compound. Measured genes include WNT target Axin2 (FIG. 3A), lacrimal duct cell marker Krt7 (FIG. 3B), and lacrimal acinar cell marker Mist1 (FIG. 3C).

FIG. 4 depicts a graph quantifying organoid outgrowth by luminescence in a 7-day WNT mimetic screen in acinar cell organoid cultures. Starting material was lacrimal gland acinar cell organoid cells. Medium contained RSPO1 alone (control), or the addition of a WNT mimetic at 5 nM concentration.

FIG. 5 depicts an optical micrograph showing organoid morphology at the end of WNT mimetic screen, in control (left panel) or L6-F12578 (right panel). Solid budding morphology in high WNT is shared with outgrowth from primary tissue and indicative of acinar cell identity. Scale bar is 200 μm.

FIG. 6 depicts a graph quantifying viable organoid cells by luminescence (in relative light units (RLU)) in organoid cells expanded for 7 days at several doses of L6-F127 WNT mimetic (0.05 nM to 5 nM) with or without RSPO1 (500 ng/mL).

FIG. 7 depicts of a schematic representation of the experimental design to measure acute WNT target gene induction at 24 hours and 48 hours post exposure.

FIG. 8 depicts a representative optical micrograph image of organoid cultures at the moment of WNT stimulus at day 7 post plating. Scale bar is 200 μm.

FIG. 9 depicts a graph quantifying WNT target Axin2 expression levels by qPCR at 24 hours (left) and 48 hours (right) post-induction. Expression for different conditions were normalized to actinB expression and relative to control (no WNT mimetic).

FIG. 10 depicts an image of two non-dry-eye human lacrimal glands, which were used in explant experiments.

FIGS. 11A and 11B depicts optical micrographs of human lacrimal gland cell cultures after dissociation (FIG. 11A) and after 24 hours in acinar cell medium (FIG. 11B). Scale bar in both FIGS. 11A and 11B is 200 μm.

FIG. 12 depicts a quantification of Axin2 expression levels by qPCR at 24 hours after start of explant culture in RSPO1 alone (control) or RSPO1+WNT mimetic at 5 nM. Expression for different conditions was normalized to actinB expression and relative to control (no WNT mimetic).

FIG. 13 depicts WNT receptor expression levels determined by in situ hybridization in naïve mouse lacrimal gland tissue. Optical micrograph histology images with probe signal in pink, scale bar is 100 μm.

FIG. 14 depicts WNT receptor expression levels determined by in situ hybridization in healthy human lacrimal gland tissue. Optical micrograph histology images with probe signal in pink, scale bar is 100 μm.

FIG. 15 depicts camera-based fluorescence microscope image showing four samples of naïve mouse lacrimal cells: a control (RSPO1 only) sample and three WNT mimetic samples after 14 days exposure (10 mpk, IP, two times a week) stained with 4′,6-diamidino-2-phenylindole (DAPI) and anti-Ki67, marker of proliferative cells (green).

FIG. 16 depicts a graph quantifying the relative weight (lacrimal gland weight/body weight) of the lacrimal gland following 14 days' treatment with various WNT mimetics. (WNT mimetic 3mpk, RSPO2 0.1 mpk, IP, twice a week). Figure legend is left to right in graph.

FIG. 17 depicts a schematic model of an in vivo mouse experiment wherein dry-eye disease was modeled by local injection of IL1a. Red represents the ipsilateral side injected with recombinant IL-1α and WNT mimetic (or control anti-GFP). Black represents the contralateral control side.

FIGS. 18A and 18B depict graphs quantifying expression of WNT target genes Axin2 (FIG. 18A) and Rnf43 (FIG. 18B) after exposure to IL1a+WNT mimetic and IL1a+anti-GFP for 8 hours and 24 hours. Expression for different conditions was normalized to actinB expression and relative to control (8-hours exposure to GFP).

FIG. 19 depicts average tear volume secretion on the ipsilateral side over a five-day time course for four experimental conditions: (1) local administration of WNT mimetic (10 μg intra-lacrimal gland); (2) local administration of anti-GFP (10 μg intra-lacrimal gland); (3) systemic administration of WNT mimetic (200 μg IP); and (4) systemic administration of anti-GFP (200 μg IP).

FIG. 20 depicts a bar graph quantifying pathology scoring of atrophy/degeneration and of inflammation in mice 3 days following lacrimal gland injection with IL1a and four different experimental treatment conditions: (1) local administration of WNT mimetic (10 μg intra-lacrimal gland); (2) local administration of anti-GFP (10 μg intra-lacrimal gland); (3) systemic administration of WNT mimetic (200 μg IP); and (4) systemic administration of anti-GFP (200 μg IP). Figure legend is shown left to right for each category.

FIGS. 21A and 21B depict optical micrograph images of lacrimal gland sections stained with hematoxylin and eosin (H&E) stain from mice injected with IL1a+WNT mimetic and IL1a+anti-GFP. Scale bar in both images is 100 μm.

FIG. 22 shows salivary gland weight (in grams) after 14 days of biweekly treatment with WNT mimetics at 3 mg/kg. Figure legend is shown left to right in graph.

FIG. 23 shows salivary gland histology after two weeks of 3 mg/kg dosing of various WNT mimetics. Panel A represents images of salivary gland histology by HE staining per treatment group at day 14. Pan B shows staining for Ki67 (proliferation marker) in brown per treatment group at day 14. Scale bars 200 μm.

FIGS. 24A and 24B show salivary gland histology after two week 10 mg/kg dosing. FIG. 24A shows representative images of salivary gland histology by HE staining per treatment group at day 14. FIG. 24B shows quantification of mucinous acini area (white) over serous acini area by Image J per treatment group. Scale bar 200 μm.

FIG. 25 is a graph representing the salivary gland weight (in grams) after two-week dosing with RSPO2-Fc at various concentrations. Figure legend is shown left to right in graph.

FIGS. 26A and 26B show murine salivary gland organoid expansion. FIG. 26A provides brightfield images of salivary gland organoids expansion from primary tissue at day 7 treated with RSPO1 or RSPO1+L-F12578. Scale bars 200 μm. FIG. 26B shows the dose dependent mouse salivary gland organoid expansion measured as cell viability at day 7 for WNT mimetics with different FZD specificities.

FIG. 27 shows murine salivary gland organoid WNT receptor profile. Gene expression levels of Fzd and Lrp genes in mouse submandibular gland organoids measured by quantitative PCR.

FIGS. 28A and 28B show the effect of treatment on Lupus mouse salivary gland. FIG. 28A provides salivary gland weight (in grams) for control (MRL/MpJ) and lupus (MRL-lpr) mice treated with Anti-GFP or L-F12578 for two weeks. FIG. 28B shows images of salivary gland histology by HE staining per treatment group for lupus and control mice at day 14. Scale bars 200 μm.

FIG. 29 shows WNT target gene Axin2 expression 24 h after local injection in IL-1α model. All treatment groups 10 μg injection. Data normalized to anti-GFP control group. Significant elevation for positive control L-F12578 and 1SH1-03.

FIG. 30 shows WNT target gene Axin2 expression 24 h after local injection in IL-1α model. All treatment groups except anti-GFP control at three different doses: 10, 50 or 150 μg injection. Data normalized to anti-GFP control group. Significant elevation all 1SH1-03 groups and the high dose 1SH1-26 groups.

FIG. 31 shows tear volume measurements using phenol red thread in the IL-1a model. Data represents an average of twelve animals. Significant tear volume elevation in several treatment groups on day 2 and 3 compared to Anti-GFP control. At day 2, the lines from top to bottom correspond to: L-F12578, 1SH1-03, 1SH1-36, 1SH1-26, and anti-GFP.

FIG. 32 illustrates WNT target gene Axin2 expression 3 days after local injection in IL-1a model. 1SH1-03 treatment groups at two different doses: 10 or 150 μg injection. Data normalized to anti-GFP control group. Significant elevation of Axin2 1SH1-03 high dose on day 3.

FIG. 33 shows tear volume measurements using phenol red thread in the IL-1α model. Data represents an average of twelve animals. Significant tear volume elevation in several treatment groups on day 2, 3, 4 compared to Anti-GFP control. At day 3, the lines from top to bottom are: L-F12578, 1SH1-36, 1SH1-03, 1SH1-26, and anti-GFP.

FIGS. 34A and 34B show duct ligation model. FIG. 34A is a schematic representation of lacrimal gland duct ligation on the ipsilateral side (blue). Other side (contralateral, red) left as control. FIG. 34B is a study timeline of duct ligation model; 3-day closure and tear volume measurements and take down after ligation removal.

FIGS. 35A and 35B show duct ligation damage. FIG. 35A shows tear volume measurements using phenol red thread after 3 days of duct closure. Ligation results in a strong decrease of tear volume that recovers over 2-3 weeks. At day 0, the top line is contralateral, and the bottom line is ipsilateral. FIG. 35B shows representative images of lacrimal gland histology following duct ligation. Severe atrophy on day 7 that slowly recovers on day 14 and day 21 compared to control.

FIG. 36 shows WNT target gene Axin2 expression at 24 h in duct ligation model, treatment after 3-day duct closure. Treatment groups at two different doses: 10 or 100 μg injection. Data normalized to anti-GFP control group. Significant elevation of Axin2 1SH1-03 and positive control L-F12578.

FIG. 37 tear volume measurements for one week using phenol red thread after 3 days of duct closure. Significant elevation of tear volume on day 7 in the positive control L-F12578 and 100 μg of 1SH1-03. At day 7, the lines from top to bottom are: L-F12578 (10 ug), 1SH1-03 (100 ug), 1SH1-03 (10 ug), and anti-GFP.

FIG. 38 shows the quantification of proliferative acinar cells (Mist1+Ki67+) in the ipsilateral and contralateral glands on day 7 of duct ligation study (from FIG. 34). Increase in proliferative cells in ipsilateral side and high dose 1SH1-03 contralateral side. For each set of bars, the bars from left to right are: GFP, L-12578 (10 ug), 1SH1-03 (10 ug), and 1SH1-03 (100 ug).

FIG. 39 shows tear volume measurements for two weeks using phenol red thread after 3 days of duct closure. Significant elevation of tear volume after day 7 in the positive control L-F12578 and 1SH1-03. At the last timepoint, the lines from top to bottom are: L-F12578 (10 ug), 1SH1-03 (100 ug), 1SH1-03 (10 ug), and anti-GFP.

FIG. 40 shows quantification of proliferative acinar cells (Mist1+Ki67+) in the ipsilateral and contralateral glands on day 14 of duct ligation study (from FIG. 39). Increase in proliferative cells in ipsilateral side and high dose 1SH1-03 contralateral side. For each set of bars, the bars from left to right correspond to: GFP, G211-18R5, 1SH1-03 (10 ug), and 1SH-03 (100 ug).

FIGS. 41A and 41B show in vitro proliferation of acinar cells. FIG. 41A shows representative images of lacrimal gland cells treated with control or 10 nM 1SH1-03. WNT activation using 1SH1-03 results in larger organoids on day 7. FIG. 41B shows cell viability quantification of organoids on day 7 expanded in control medium or different doses of 1SH1-03. Dose-response effect with significantly more cells with 1SH1-03.

FIG. 42 shows the quantification of salivary gland proliferation in animals upon two-week systemic dosing in animals. Administration of L-F12578 has more proliferative epithelial cells (Ki67+/ECad+) on Day 7 compared to the control group and other timepoints. Vehicle is left, and L-F12578 is right for each timepoint. For each timepoint, the left bar is Vehicle, and the right bar is L-F12578.

FIG. 43 shows the quantification of salivary gland tissue weight in animals upon two-week systemic dosing in animals. The administration of L-F12578 significantly elevates organ weight at day 7, 9, 11 and 14 compared to control. Vehicle is left, and L-F12578 is right for each timepoint. For each timepoint, the left bar is Vehicle, and the right bar is L-F12578.

It will be readily apparent to persons having ordinary skill in the art that the above figures are illustrative only and do not limit the scope of the instant disclosure.

### DETAILED DESCRIPTION

### I. Definitions

As used herein, including the appended claims, the singular forms of words such as “a,” “an,” and “the,” include their corresponding plural references unless the context clearly dictates otherwise.

All references cited herein are incorporated by reference to the same extent as if each individual publication, patent application, or patent, was specifically and individually indicated to be incorporated by reference.

“Activity” of a molecule may describe or refer to the binding of the molecule to a ligand or to a receptor, to catalytic activity, to the ability to stimulate gene expression, to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” may also mean specific activity, e.g., [catalytic activity]/[mg protein], or [immunological activity]/[mg protein], or the like.

The terms “administering” or “introducing” or “providing”, as used herein, refer to delivery of a composition to a cell, to cells, tissues and/or organs of a subject, or to a subject. Such administering or introducing may take place in vivo, in vitro or ex vivo.

As used herein, the term “antibody” means an isolated or recombinant binding agent that comprises the necessary variable region sequences to specifically bind an antigenic epitope. Therefore, an antibody is any form of antibody or fragment thereof that exhibits the desired biological activity, e.g., binding the specific target antigen. Thus, it is used in the broadest sense and specifically covers monoclonal antibodies (including full-length monoclonal antibodies), polyclonal antibodies, human antibodies, humanized antibodies, chimeric antibodies, nanobodies, diabodies, multispecific antibodies (e.g., bispecific antibodies), and antibody fragments including, but not limited to, scFv, Fab, and Fab2, so long as they exhibit the desired biological activity.

“Antibody fragments” comprise a portion of an intact antibody, for example, the antigen-binding or variable region of the intact antibody. Examples of antibody fragments include Fab, Fab′, F(ab′)2, and Fv fragments; diabodies; linear antibodies (e.g., Zapata et al., Protein Eng. 8(10): 1057-1062 (1995)); single-chain antibody molecules (e.g., scFv); and multispecific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

The term “antigen” refers to a molecule or a portion of a molecule capable of being bound by a selective binding agent, such as an antibody, and 30 additionally capable of being used in an animal to produce antibodies capable of binding to an epitope of that antigen. In certain embodiments, a binding agent (e.g., a WNT surrogate molecule or binding region thereof, or a WNT antagonist) is said to specifically bind an antigen when it preferentially recognizes its target antigen in a complex mixture of proteins and/or macromolecules.

The term “antigen-binding fragment” as used herein refers to a polypeptide fragment that contains at least one complementarity-determining region (CDR) of an immunoglobulin heavy and/or light chain, or of a VTHsdAb (single domain antibody) or Nanobody® (Nab), that binds to the antigen of interest, in particular to one or more Fzd receptors, or to LRP5 and/or LRP6. In this regard, an antigen-binding fragment of the herein described antibodies may comprise 1, 2, 3, 4, 5, or all 6 CDRs of a VH and VL from antibodies that bind one or more Fzd receptors or LRP5 and/or LRP6.

As used herein, the terms “biological activity” and “biologically active” refer to the activity attributed to a particular biological element in a cell. For example, the “biological activity” of a WNT agonist, or fragment or variant thereof refers to the ability to mimic or enhance WNT signals. As another example, the biological activity of a polypeptide or functional fragment or variant thereof refers to the ability of the polypeptide or functional fragment or variant thereof to carry out its native functions of, e.g., binding, enzymatic activity, etc. In some embodiments, a functional fragment or variant retains at least 20%, at least 30%, at least 40%, at least 50%, at least 60%, at least 70%, at least 80%, at least 90%, or at least 100% of an activity of the corresponding native protein or nucleic acid. As a third example, the biological activity of a gene regulatory element, e.g., promoter, enhancer, Kozak sequence, and the like, refers to the ability of the regulatory element or functional fragment or variant thereof to regulate, i.e., promote, enhance, or activate the translation of, respectively, the expression of the gene to which it is operably linked.

The term “bifunctional antibody,” as used herein, refers to an antibody that comprises a first arm having a specificity for one antigenic site and a second arm having a specificity for a different antigenic site, i.e., the bifunctional antibodies have a dual specificity.

“Bispecific antibody” is used herein to refer to a full-length antibody that is generated by quadroma technology (see Milstein et al., Nature, 305(5934): 537-540 (1983)), by chemical conjugation of two different monoclonal antibodies (see Staerz et al., Nature, 314(6012): 628-631 (1985)), or by knob-into-hole or similar approaches, which introduce mutations in the Fc region (see Holliger et al., Proc. Natl. Acad. Sci. USA, 90(14): 6444-6448 (1993)), resulting in multiple different immunoglobulin species of which only one is the functional bispecific antibody. A bispecific antibody binds one antigen (or epitope) on one of its two binding arms (one pair of HC/LC), and binds a different antigen (or epitope) on its second arm (a different pair of HC/LC). By this definition, a bispecific antibody has two distinct antigen-binding arms (in both specificity and CDR sequences) and is monovalent for each antigen to which it binds.

By “comprising,” it is meant that the recited elements are required in, for example, the composition, method, kit, etc., but other elements may be included to form the, for example, composition, method, kit etc. within the scope of the claim. For example, an expression cassette “comprising” a gene encoding a therapeutic polypeptide operably linked to a promoter is an expression cassette that may include other elements in addition to the gene and promoter, e.g., poly-adenylation sequence, enhancer elements, other genes, linker domains, etc.

By “consisting essentially of,” it is meant a limitation of the scope of the, for example, composition, method, kit, etc., described to the specified materials or steps that do not materially affect the basic and novel characteristic(s) of the, for example, composition, method, kit, etc. For example, an expression cassette “consisting essentially of” a gene encoding a therapeutic polypeptide operably linked to a promoter and a polyadenylation sequence may include additional sequences, e.g., linker sequences, so long as they do not materially affect the transcription or translation of the gene. As another example, a variant, or mutant, polypeptide fragment “consisting essentially of” a recited sequence has the amino acid sequence of the recited sequence plus or minus about 10 amino acid residues at the boundaries of the sequence based upon the full length naïve polypeptide from which it was derived, e.g., 10, 9, 8, 7, 6, 5, 4, 3, 2 or 1 residue less than the recited bounding amino acid residue, or 1, 2, 3, 4, 5, 6, 7, 8, 9, or 10 residues more than the recited bounding amino acid residue.

By “consisting of,” it is meant the exclusion from the composition, method, or kit of any element, step, or ingredient not specified in the claim. For example, a polypeptide or polypeptide domain “consisting of” a recited sequence contains only the recited sequence.

A “control element” or “control sequence” is a nucleotide sequence involved in an interaction of molecules that contributes to the functional regulation of a polynucleotide, including replication, duplication, transcription, splicing, translation, or degradation of the polynucleotide. The regulation may affect the frequency, speed, or specificity of the process, and may be enhancing or inhibitory in nature. Control elements known in the art include, for example, transcriptional regulatory sequences such as promoters and enhancers. A promoter is a DNA region capable under certain conditions of binding RNA polymerase and initiating transcription of a coding region usually located downstream (in the 3′ direction) from the promoter.

An “expression vector” is a vector, e.g., plasmid, minicircle, viral vector, liposome, and the like as discussed herein or as known in the art, comprising a region which encodes a gene product of interest, and is used for effecting the expression of the gene product in an intended target cell. An expression vector also comprises control elements, e.g., promoters, enhancers, untranslated regions (UTRs), miRNA targeting sequences, etc., operatively linked to the encoding region to facilitate expression of the gene product in the target. The combination of control elements and a gene or genes to which they are operably linked for expression is sometimes referred to as an “expression cassette,” a large number of which are known and available in the art or can be readily constructed from components that are available in the art.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

The terms “individual,” “host,” “subject,” and “patient” are used interchangeably herein, and refer to a mammal, including, but not limited to, human and non-human primates, including simians and humans; mammalian sport animals (e.g., horses); mammalian farm animals (e.g., sheep, goats, etc.); mammalian pets (dogs, cats, etc.); and rodents (e.g., mice, rats, etc.).

“Humanized” antibodies or fragments thereof refers to antibodies or fragments thereof from non-human species whose protein sequences have been modified to increase their similarity to antibody variants produced naturally in humans. The process of “humanization” is usually applied to monoclonal antibodies developed for administration to humans.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), single domain antibodies (sdAbs, also known as Nanobodies), variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including WNT surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

The term “native” or “wild-type” as used herein refers to a nucleotide sequence, e.g., gene, or gene product, e.g., RNA or protein, that is present in a wild-type cell, tissue, organ, or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 50% or more, 60% or more, or 70% or more with a full-length native polynucleotide sequence, e.g., an identity of 75% or 80% or more, such as 85%, 90%, or 95% or more, for example, 98% or 99% identity with the full-length native polynucleotide sequence. As another example, a variant may be a polypeptide having a sequence identity of 70% or more with a full-length native polypeptide sequence, e.g., an identity of 75% or 80% or more, such as 85%, 90%, or 95% or more, for example, 98% or 99% identity with the full-length native polypeptide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

The term “polynucleotide” refers to a polymeric form of nucleotides of any length, including deoxyribonucleotides or ribonucleotides, or analogs thereof. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double-stranded and single-stranded molecules. Unless otherwise specified or required, any embodiment of the invention described herein that is a polynucleotide encompasses both the double-stranded form and each of two complementary single-stranded forms known or predicted to make up the double-stranded form.

A polynucleotide or polypeptide has a certain percent “sequence identity” to another polynucleotide or polypeptide, meaning that, when aligned, that percentage of bases or amino acids are the same when comparing the two sequences. The terms “identical” or “identity” when used in the context of two or more nucleic acids or polypeptide sequences, refer to the number or percentage of residues that are the same in a sequence of interest and a reference sequence. The percentage can be calculated by optimally aligning the sequence of interest to the reference sequence; comparing the two sequences over the entire length of the reference sequence; determining the number of positions at which the identical amino acid residue or nucleic acid base occurs in both sequences to yield the number of matched positions; dividing the number of matched positions by the total number of positions in the reference sequence adjusted by adding the number of gap positions introduced into the reference sequence in generating the alignment; and multiplying the result by 100 to yield the percentage of sequence identity. When comparing DNA and RNA, thymine (T) and uracil (U) can be considered equivalent Sequence “identity” may be determined by using the stand-alone executable BLAST engine program for blasting two sequences (bl2seq), which can be retrieved from the National Center for Biotechnology Information (NCBI) ftp site or over the worldwide web at ncbi.nlm.nih.gov/BLAST/, using the default parameters (Tatusova and Madden, FEMS Microbiol Lett., 1999, 174, 247-250; which is incorporated herein by reference in its entirety).

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species or cell-type specific, tissue-specific, or species specific. Promoters may be “constitutive,” meaning continually active, or “inducible,” meaning the promoter can be activated or deactivated by the presence or absence of biotic or abiotic factors. Also included in the nucleic acid constructs or vectors of the invention are enhancer sequences that may or may not be contiguous with the promoter sequence. Enhancer sequences influence promoter-dependent gene expression and may be located in the 5′ or 3′ regions of the native gene.

“Recombinant,” as applied to a polynucleotide means that the polynucleotide is the product of various combinations of cloning, restriction or ligation steps, and other procedures that result in a construct that is distinct from a polynucleotide found in nature.

As used herein “Sjögren's syndrome” or “Sjogren's syndrome” is a chronic autoimmune disease affecting the lacrimal and salivary glands, defined according to customary international diagnostic criteria such as described in Vitali et al (2002) Ann. Rheum. Dis. 61:554. Sjögren's syndrome is one of two major subclasses of aqueous-deficient dry-eye diseases, distinguished from non-Sjögren's syndrome type.

As used herein, “SWAP™” (Surrozen WNT-signal activating proteins) refers to WNT mimetic compounds comprising engineered bi-specific full-length immunoglobulin-G (IgG) antibodies that, like WNT proteins, directly activate the canonical WNT-signaling pathway in a target tissue, e.g., lacrimal gland tissue.

As used herein, “SWEETS™” (Surrozen WNT-signal enhancer engineered for tissue specificity) refers to antibody-based R-spondin mimetic compounds as described in US20200048324.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal and includes: (a) inhibiting the disease, i.e., arresting or slowing its development; or (b) relieving the disease, i.e., causing regression or reduction of the disease or reducing the severity of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The practice of the present invention will employ, unless otherwise indicated, conventional techniques of cell biology, molecular biology techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

All publications mentioned herein are incorporated herein by reference to disclose and describe the methods and/or materials in connection with which the publications are cited. It is understood that the present disclosure supersedes any disclosure of an incorporated publication to the extent there is a contradiction.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Unless otherwise indicated, all terms used herein have the same meaning as they would to one skilled in the art and the practice of the present invention will employ, conventional techniques of microbiology and recombinant DNA technology, which are within the knowledge of those of skill of the art.

### II. General

The present invention provides methods of modulating WNT signals to treat lacrimal gland disorders due to, including but not limited to, Sjögren's syndrome disorder, chronic graft versus host disease (cGHVD), rheumatoid arthritis (RA), Stephen's Johnson syndrome, Ocular Rosacea, chemotherapy, radiation oncology treatments, diabetes, roasacea, lupus, etc.

WNT (“Wingless-related integration site” or “Wingless and Int-1” or “Wingless-Int”) ligands and their signals play key roles in the control of development, homeostasis and regeneration of many essential organs and tissues, including bone, liver, skin, stomach, intestine, kidney, central nervous system, mammary gland, taste bud, ovary, cochlea, lung, and many other tissues (reviewed, e.g., by Clevers, Loh, and Nusse, 2014; 346:1248012). Modulation of WNT signaling pathways has potential for treatment of degenerative diseases and tissue injuries.

One of the challenges for modulating WNT signaling as a therapeutic is the existence of multiple WNT ligands and WNT receptors, Frizzled 1-10 (Fzd1-10), with many tissues expressing multiple and overlapping Fzds. Canonical WNT signals also involve Low-density lipoprotein (LDL) receptor-related protein 5 (LRP5) or Low-density lipoprotein (LDL) receptor-related protein 6 (LRP6) as co-receptors, which are broadly expressed in various tissues, in addition to Fzds. R-spondins 1-4 are a family of ligands that amplify WNT signals. Each of the R-spondins work through a receptor complex that contains Zinc and Ring Finger 3 (ZNRF3) or Ring Finger Protein 43 (RNF43) on one end and a Leucine-rich repeat-containing G-protein coupled receptor 4-6 (LGR4-6) on the other (reviewed, e.g., by Knight & Hankenson 2014, Matrix Biol.; 37: 157-161). R-spondins might also work through additional mechanisms of action. ZNRF3 and RNF43 are two membrane-bound E3 ligases specifically targeting WNT receptors (Fzd1-10 and LRP5 or LRP6) for degradation. Binding of an R-spondin to ZNRF3/RNF43 and LGR4-6 causes clearance or sequestration of the ternary complex, which removes E3 ligases from WNT receptors and stabilizes WNT receptors, resulting in enhanced WNT signals. Each R-spondin contains two Furin domains (1 and 2), with Furin domain 1 binding to ZNRF3/RNF43, and Furin domain 2 binding to LGR4-6. Fragments of R-spondins containing Furin domains 1 and 2 are sufficient for amplifying WNT signaling. While R-spondin effects depend on WNT signals, since both LGR4-6 and ZNRF3/RNF43 are widely expressed in various tissues, the effects of R-spondins are not tissue-specific.

Activating WNT signaling by a WNT agonist may be used for the treatment of a variety of lacrimal gland diseases and disorders, including dry-eye diseases. Similarly, amplifying WNT signaling by RSPO or an RSPO mimetic may be used for the treatment of a variety of lacrimal gland diseases and disorders, including various dry eye and salivary gland diseases. A WNT agonist molecule may also be used for the treatment of dry eye and salivary gland disorders. In particular, active WNT signaling can provide a major stem cell maintenance signal and plays a key role in regulating regeneration of the acinar cells, e.g., in salivary glands.

### III. Engineered WNT Agonists

The present disclosure provides engineered WNT agonists and contemplates the use of engineered WNT agonists to stimulate, agonize, or promote WNT signaling, e.g., through the canonical WNT/β-catenin signaling pathway. Such engineered WNT agonists may also be referred to as WNT/β-catenin signaling agonists or Wnt mimetics.

The disclosure provides engineered Wnt mimetics with drug-like properties, particularly in the form of recombinant, bi-specific antibodies that bring together Fzd and Lrp to stimulate signaling, mimicking endogenous Wnt ligands. The Wnt mimetics of the disclosure may freely diffuse, access damaged tissues and guide tissue repair where Wnt signals are needed. The disclosure also provides Wnt mimetics that are capable of repairing damaged lacrimal or salivary gland tissue without being combined with RSPO.

In some embodiments, the WNT/β-catenin signaling antagonist or agonist can include binding agents or epitope binding domains that bind one or more Fzd receptors and inhibit or enhance WNT signaling. In certain embodiments, the agent or antibody specifically binds to the cysteine-rich domain (CRD) within the human frizzled receptor(s) to which it binds. Additionally, antagonistic binding agents containing epitope binding domains against LRP can also be used. In some embodiments, the WNT/β-catenin antagonist possesses binding agents or epitope binding domains that bind E3 ligases ZNRF3/RNF43 and one or more FZD receptors or one or more LRP co-receptors to promote the degradation of FZD or LRP receptors, and this molecule can also contain a binding domain that binds a cell type specific epitope for targeting. The E3 ligase agonist antibodies or fragments thereof can be single molecules or combined with other WNT antagonists, e.g., Fzd receptor antagonists, LRP receptor antagonists, etc.

As is well known in the art, an antibody is an immunoglobulin molecule capable of specific binding to a target such as a carbohydrate, polynucleotide, lipid, polypeptide, etc., through at least on epitope binding domain, located on the variable region of the immunoglobulin molecule. As used herein, the term encompasses not only intact polyclonal or monoclonal antibodies, but also fragments thereof containing epitope binding domains (e.g., dAb, Fab, Fab′, (F(ab′)2, Fv, single chain (scFv), VHH (i.e., Nanobodies®) or single domain antibodies (sdAb), DVD-Igs (also known as Fv-Igs), synthetic variants thereof, naturally occurring variants, fusion proteins comprising and epitope binding domain, humanized antibodies, chimeric antibodies, and any other modified configuration of the immunoglobulin molecule that comprises an antigen-binding site or fragment (epitope recognition site) of the required specificity. “Diabodies,” multivalent or multispecific fragments constructed by gene fusion (WO94/13804; P. Holliger et al., Proc. Natl. Acad. Sci. USA 90 6444-6448, 1993) are also a particular form of antibody contemplated herein. Minibodies comprising a scFv joined to a CH3 domain are also included herein (S. Hu et al., Cancer Res., 56, 3055-3061, 1996). See e.g., Ward, E. S. et al., Nature 341, 544-546 (1989); Bird et al., Science, 242, 423-426, 1988; Huston et al., Proc. Natl. Acad. Sci. USA, 85, 5879-5883, 1988); PCT/US92/09965; WO94/13804; P. Holliger et al., Proc. Natl. Acad. Sci. USA 90 6444-6448, 1993; Y. Reiter et al., Nature Biotech, 14, 1239-1245, 1996; S. Hu et al., Cancer Res., 56, 3055-3061, 1996; C. Bever et al., Anal Bioanal Chem. 2016 September; 408(22); 5985-6002.

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present disclosure can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

In certain embodiments, single chain Fv or scFV antibodies are contemplated. For example, Kappa bodies (Ill et al., Prot. Eng. 10: 949-57 (1997)); minibodies (Martin et al., EMBO J 13: 5305-9 (1994)); diabodies (Holliger et al., Proc. Nat. Acad. Sci. 90: 6444-8 (1993)); or Janusins (Traunecker et al., EMBO J 10: 3655-59 (1991) and Traunecker et al., Int. J. Cancer Suppl. 7: 51-52 (1992)), may be prepared using standard molecular biology techniques following the teachings of the present application with regard to selecting antibodies having the desired specificity. In still other embodiments, bispecific or chimeric antibodies may be made that encompass the ligands of the present disclosure. For example, a chimeric antibody may comprise CDRs and framework regions from different antibodies, while bispecific antibodies may be generated that bind specifically to one or more Fzd receptors through one binding domain and to a second molecule through a second binding domain. These antibodies may be produced through recombinant molecular biological techniques or may be physically conjugated together.

A single chain Fv (scFv) polypeptide is a covalently linked VH:VL heterodimer which is expressed from a gene fusion including VH- and VL-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an scFv molecule which will fold into a three-dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, an antibody as described herein is in the form of a diabody. Diabodies are multimers of polypeptides, each polypeptide comprising a first domain comprising a binding region of an immunoglobulin light chain and a second domain comprising a binding region of an immunoglobulin heavy chain, the two domains being linked (e.g., by a peptide linker) but unable to associate with each other to form an antigen binding site: antigen binding sites are formed by the association of the first domain of one polypeptide within the multimer with the second domain of another polypeptide within the multimer (WO94/13804).

A dAb fragment of an antibody consists of a VH domain (Ward, E. S. et al., (1989) Nature 341:544-546).

Where bispecific antibodies are to be used, these may be conventional bispecific antibodies, which can be manufactured in a variety of ways (Holliger, P. & Winter G., Curr. Opin. Biotech. 4, 446-449 (1993)), e.g., prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (J. B. B. Ridgeway et al., Protein Eng., 9, 616-621 (1996)).

In certain embodiments, the antibodies described herein may be provided in the form of a UniBody®. A UniBody® is an IgG4 antibody with the hinge region removed (see GenMab Utrecht, The Netherlands; see also, e.g., US2009/0226421). This proprietary antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions aredenoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures-regardless of the precise CDR amino acid sequence. Further, certain FR residues are known to participate in non-covalent interdomain contacts which stabilize the interaction of the antibody heavy and light chains.

A “monoclonal antibody” refers to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (scFv), Nanobodies®, variants thereof, fusion proteins comprising an antigen-binding fragment of a monoclonal antibody, humanized monoclonal antibodies, chimeric monoclonal antibodies, and any other modified configuration of the immunoglobulin molecule that comprises an antigen-binding fragment (epitope recognition site) of the required specificity and the ability to bind to an epitope, including WNT surrogate molecules disclosed herein. It is not intended to be limited as regards the source of the antibody or the manner in which it is made (e.g., by hybridoma, phage selection, recombinant expression, transgenic animals, etc.). The term includes whole immunoglobulins as well as the fragments etc. described above under the definition of “antibody”.

In certain embodiments, the antibodies of the present disclosure may take the form of a single domain antibody (sdAb). SdAb technology was originally developed following the discovery and identification that camelidae (e.g., camels, alpacas, and llamas) possess fully functional antibodies that consist of heavy chains only and therefore lack light chains. These heavy-chain only antibodies contain a single variable domain (VHH) and two constant domains (CH2, CH3). The cloned and isolated single variable domains have full antigen binding capacity and are very stable. These single variable domains, with their unique structural and functional properties, form the basis of sdAbs. SdAb are encoded by single genes and are efficiently produced in almost all prokaryotic and eukaryotic hosts, e.g., E. coli (see, e.g., U.S. Pat. No. 6,765,087), molds (for example Aspergillus or Trichoderma) and yeast (for example Saccharomyces, Kluyvermyces, Hansenula, or Pichia (see, e.g., U.S. Pat. No. 6,838,254). The production process is scalable and multi-kilogram quantities of sdAbs have been produced. SdAbs may be formulated as a ready-to-use solution having a long shelf life. The Nanoclone® method (see, e.g., WO 06/079372) is a proprietary method for generating sdAbs against a desired target, based on automated high-throughput selection of B-cells. SdAb are single-domain antigen-binding fragments of camelid-specific heavy-chain only antibodies. sdAb, also referred to as VHH antibodies, typically have a small size of around 15 kDa. See C. Bever et al., Anal Bioanal Chem. 2016 September; 408(22); 5985-6002.

Another antibody fragment contemplated is a dual-variable domain-immunoglobulin (DVD-Ig or Fv-Ig) is an engineered protein that combines the function and specificity of two monoclonal antibodies in one molecular entity. An Fv-Igis designed as an IgG-like molecule, except that each light chain and heavy chain contains two variable domains in tandem through a short peptide linkage, instead of one variable domain in IgG. The fusion orientation of the two variable domains and the choice of linker sequence are critical to functional activity and efficient expression of the molecule. An Fv-Ig can be produced by conventional mammalian expression systems as a single species for manufacturing and purification. An Fv-Ig has the specificity of the parental antibodies, is stable in vivo, and exhibits IgG-like physicochemical and pharmacokinetic properties. Fv-Igs and methods for making them are described in Wu, C., et al., Nat Biotech, 25:1290-1297 (2007)).

In certain embodiments, the antibodies or antigen-binding fragments thereof as disclosed herein are humanized. This refers to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio, A. F. et al., (1989) Proc Natl Acad Sci USA 86:4220-4224; Queen et al., PNAS (1988) 86:10029-10033; Riechmann et al., Nature (1988) 332:323-327). Illustrative methods for humanization of the anti-Fzd or LRP antibodies disclosed herein include the methods described in U.S. Pat. No. 7,462,697.

Another approach focuses not only on providing human-derived constant regions but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato, K., et al., (1993) Cancer Res 53:851-856; Riechmann, L., et al., (1988) Nature 332:323-327; Verhoeyen, M., et al., (1988) Science 239:1534-1536; Kettleborough, C. A., et al., (1991) Protein Engineering 4:773-3783; Maeda, H., et al., (1991) Human Antibodies Hybridoma 2:124-134; Gorman, S. D., et al., (1991) Proc Natl Acad Sci USA 88:4181-4185; Tempest, P. R., et al., (1991) Bio Technology 9:266-271; Co, M. S., et al., (1991) Proc Natl Acad Sci USA 88:2869-2873; Carter, P., et al., (1992) Proc Natl Acad Sci USA 89:4285-4289; and Co, M. S. et al., (1992) J Immunol 148:1149-1154. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies of the present disclosure may be chimeric antibodies. In this regard, a chimeric antibody is comprised of an antigen-binding fragment of an antibody operably linked or otherwise fused to a heterologous Fc portion of a different antibody. In certain embodiments, the heterologous Fc domain is of human origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

The structures and locations of immunoglobulin CDRs and variable domains may be determined by reference to Kabat, E. A. et al., SEQUENCES OF PROTEINS OF IMMUNOLOGICAL INTEREST, 4th Edition, US Department of Health and Human Services. 1987, and updates thereof, now available on the Internet (immuno.bme.nwu.edu).

In certain embodiments, the antagonist or agonist binding agent binds with a dissociation constant (KD) of about 1 μM or less, about 100 nM or less, about 40 nM or less, about 20 nM or less, or about 10 nM or less. For example, in certain embodiments, a FZD binding agent or antibody described herein that binds to more than one FZD, binds to those FZDs with a KD of about 100 nM or less, about 20 nM or less, or about 10 nM or less. In certain embodiments, the binding agent binds to one or more its target antigen with an EC50 of about 1 μM or less, about 100 nM or less, about 40 nM or less, about 20 nM or less, about 10 nM or less, or about 1 nM 20 or less.

The antibodies or other agents of the present invention can be assayed for specific binding by any method known in the art. The immunoassays which can be used include, but are not limited to, competitive and non-competitive assay systems using techniques such as biolayer interferometry (BLI) analysis, FACS analysis, immunofluorescence, immunocytochemistry, Western blots, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al, eds, 1994, CURRENT PROTOCOLS IN MOLECULAR BIOLOGY, Vol. 1, John Wiley & Sons, Inc., New York, which is incorporated by reference herein in its entirety).

For example, the specific binding of an antibody to a target antigen may be determined using ELISA. An ELISA assay comprises preparing antigen, coating wells of a 96 well microtiter plate with antigen, adding the antibody or other binding agent conjugated to a detectable compound such as an enzymatic substrate (e.g., horse-radish peroxidase or alkaline phosphatase) to the well, incubating for a period of time and detecting the presence of the antigen. In some embodiments, the antibody or agent is not conjugated to a detectable compound, but instead a second conjugated antibody that recognizes the first antibody or agent is added to the well. In some embodiments, instead of coating the well with the antigen, the antibody or agent can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art (see, e.g., Ausubel et al, eds, 1994, CURRENT PROTOCOLS IN MOLECULAR BIOLOGY, Vol. 1, John Wiley & Sons, Inc., New York at 11.2.1).

The binding affinity of an antibody or other agent to a target antigen and the off-rate of the antibody-antigen interaction can be determined by competitive binding assays. One example of a competitive binding assay is a radioimmunoassay comprising the incubation of labeled antigen (e.g., Fzd, LRP), or fragment or variant thereof, with the antibody of interest in the presence of increasing amounts of unlabeled antigen followed by the detection of the antibody bound to the labeled antigen. The affinity of the antibody and the binding off-rates can be determined from the data by scattered plot analysis. In some embodiments, BLI analysis is used to determine the binding on and off rates of antibodies or agents. BLI kinetic analysis comprises analyzing the binding and dissociation of antibodies from chips with immobilized antigens on their surface.

### IV. Compositions of Methods of Use of Wnt Agonists

The present description provides methods of treating lacrimal gland diseases and disorders, including but not limited to dry-eye disorders, e.g., by activation and/or regeneration of lacrimal gland acinar cells, and compositions therefor.

It will be readily understood to persons skilled in the art that lacrimal gland is homologous to Meibomian gland and accessory lacrimal glands, and the methods and compositions described herein may be applied for treatment of the Meibomian glands and/or accessory lacrimal glands in aqueous-deficient or evaporative dry eye. Meibomian glands are oil glands which line the margin of eyelids. These glands cause the eyelids to secrete oils that blend with natural tears, preventing excessive evaporation of the natural tears. Patients suffering from ocular rosacea often have Meibomian gland dysfunction (MGD) where the eyelids secrete fewer oils resulting dry eyes. MGD is thought to be the leading cause of dry eye disease. A WNT agonist molecule may also be used for the treatment of Meibomian gland disorders. In particular, active WNT signaling can potentially provide a maintenance signal to basal progenitor cells and plays a key role in regulating regeneration of the meibocytes (see, e.g., Parfitt et al (2016) Stem Cell Rep. 7:399-410). In one aspect, the present invention comprises a method of regenerating lacrimal gland acinar cells in a subject, comprising administering a WNT signaling modulator to the subject. In an embodiment, the WNT signaling modulator may be an engineered WNT signaling modulator such as a SWAP™ compound. In another embodiment, the WNT signaling modulator is an engineered WNT agonist or an engineered WNT antagonist. In yet a further embodiment, the WNT signaling modulator is a tissue-specific WNT signal enhancing molecule, such as a SWEETS™ molecule. A WNT signaling modulator may also be a combination of a WNT agonist and a tissue-specific WNT signal enhancer.

In particular embodiments of any of the methods disclosed herein, the WNT agonist is selected from those disclosed in any of the following: PCT Application Publication No. WO 2016/040895; US Application Publication No. US 2017-0306029; US Application Publication No. US 2017-0349659; PCT Application Publication No. WO 2019/126398; PCT Application Publication No. WO 2020/01030, PCT Application Publication No. WO 2021/173726, or U.S. application Ser. No. 17/806,624, all of which are herein incorporated by reference in their entireties. In particular embodiments of any of the methods disclosed herein, the tissue-specific WNT signal enhancing molecule is selected from those disclosed in any of the following: PCT Application Publication No. WO 2018/140821; US Application Publication No. US 2020-0048324; or PCT Application Publication No. WO 2020/14271, all of which are herein incorporated by reference in their entireties.

In an embodiment of the method of regenerating lacrimal gland acinar cells, the WNT signaling modulator may comprise at least one engineered bi-specific full-length IgG antibody that directly activates a canonical WNT signaling pathway. In any embodiment, the engineered WNT agonist may be selected from: (i) WNT3a; (ii) a WNT mimetic; or (iii) an β-spondin mimetic. The WNT mimetic may be a SWAP™ compound. The R-spondin mimetic may be a SWEETS™ compound. The WNT mimetics may comprise one or more polypeptides comprising or having a polypeptide sequence set forth in any of SEQ ID Nos: 1-14 isoforms and homologs thereof, and suitable expression vectors therefor. The WNT mimetics may comprise one or more polypeptides having 80% to 100% homology with any of the polypeptide sequences set forth in SEQ ID Nos 1-14. In certain embodiments, the WNT mimetic comprises two polypeptide sequences having 80% to 100% homology with any of the polypeptide sequences set forth in SEQ ID Nos 1-14. In certain embodiments, the WNT mimetic comprises two heavy chain and two light chain polypeptide sequences, each having 80% to 100% homology with any of the polypeptide sequences set forth in SEQ ID Nos 1-14. In particular embodiments, the heavy chain and light chain polypeptides present in the WNT mimetic have at least 80%, at least 90%, at least 95%, at least 98%, or 100% identity to any of following combinations: SEQ ID NOs 1 and 2; SEQ ID NOs: 3 and 4; SEQ ID NOs: 5 and 6; SEQ ID NOs: 7 and 8; or SEQ ID NOs: 9 and 10, or SEQ ID NOs: 11 and 14, or SEQ ID NOs: 12 and 14, or SEQ ID NOs: 13 and 14. In particular embodiments, the WNT mimetic has an IgG antibody structure comprising two heavy and two light chains, wherein the two heavy chains are bound to each other, and each of the light chains is bound to a different one of the two heavy chains.

In an embodiment of the method of regenerating lacrimal gland acinar cells, the WNT signaling modulator may affect expression of any one or more of: Fzd1, Fzd2, Fzd5, Fzd7, Fzd8, and Lrp6, and/or Lrp5. For example, the WNT signaling modulator may target any one or more of the group consisting of: Fzd1, Fzd2, and Fzd7; or any one or more of the group consisting of: Fzd5 and Fzd8, while also targeting Lrp6 and/or Lrp5. For another example, the WNT signaling modulator may affect expression of any one or more of: Fzd1, Fzd2, and Fzd7.

In one embodiment of the method of regenerating lacrimal gland acinar cells, the method further comprises the step of administering at least one of the group consisting of: RSPO2, RSPO2 fragment, and engineered RSPO2 mimetic.

In some embodiment the WNT signaling modulator may be a superagonist of WNT platform. This superagonist activity was observed with LRP, FZD, and RSPO fused WNT molecule acting as a robust activator of WNT signaling pathway. the WNT superagonist is selected from those disclosed in following: PCT Application Publication No. WO 2021/173726.

In any embodiment, the WNT signaling modulator may be in a concentration ≥1 nM. For example, the WNT signaling modulator may be in a concentration of 1 nM, 1.1 nM, 1.5 nM, 2.0 nM, 2.5 nM, 3.0 nM, 3.5 nM, 4.0 nM, 4.5 nM, 5.0 nM. In certain embodiments, the concentration of WNT signaling modulator may be ≥5 nM. In any embodiment, the WNT signaling modulator may be administered in a therapeutically effective amount.

In any embodiment, the subject may be a live mammal. For example, the subject may be mouse, rat, dog, cat, horse, or cow. In any embodiment, the subject may be a human patient.

In any embodiment, the WNT signaling modulator may be administered systemically or locally. For example, the WNT signaling modulator may be administered locally via aqueous eyedrop solution or local intra-lacrimal gland injection.

In another aspect, the present invention comprises a method of treating a lacrimal gland disorder in a subject, comprising administering a WNT signaling modulator to the subject. In an embodiment, the WNT signaling modulator may be an engineered WNT signaling modulator. In another embodiment, the WNT signaling modulator is an engineered WNT agonist or an engineered WNT antagonist.

In an embodiment of the method of treating a lacrimal gland disorder, the WNT signaling modulator may comprise at least one engineered bi-specific full-length IgG antibody that directly activates a canonical WNT signaling pathway. In any embodiment, the engineered WNT agonist may be selected from: (i) WNT3a; (ii) a WNT mimetic; or (iii) an R-spondin mimetic. The WNT mimetic may be a SWAP™ compound. The R-spondin mimetic may be a SWEETS™ compound.

In an embodiment of the method of treating a lacrimal gland disorder, the WNT signaling modulator may affect expression of any one or more of: Fzd1, Fzd2, Fzd5, Fzd7, Fzd8, and Lrp6 and/or Lrp5. For example, the WNT signaling modulator may target any one or more of the group consisting of: Fzd1, Fzd2, and Fzd7; or any one or more of the group consisting of: Fzd5 and Fzd8, while also targeting Lrp6 and/or Lrp5. For another example, the WNT signaling modulator may affect expression of any one or more of: Fzd1, Fzd2, and Fzd7.

In one embodiment of the method of treating a lacrimal gland disorder, the method further comprises the step of administering at least one of the group consisting of: RSPO2, RSPO2 fragment, and engineered RSPO2 mimetic.

In any embodiment of the method of treating a lacrimal gland disorder, the WNT signaling modulator may be in a concentration ≥1 nM. For example, the WNT signaling modulator may be in a concentration of 1 nM, 1.1 nM, 1.5 nM, 2.0 nM, 2.5 nM, 3.0 nM, 3.5 nM, 4.0 nM, 4.5 nM, 5.0 nM. In certain embodiments, the concentration of WNT signaling modulator may be ≥5 nM. In any embodiment, the WNT signaling modulator may be administered in a therapeutically effective amount.

In any embodiment, the subject may be a live mammal. For example, the subject may be mouse, rat, dog, cat, horse, or cow. In any embodiment, the subject may be a human patient.

In any embodiment, the WNT signaling modulator may be administered systemically or locally. For example, the WNT signaling modulator may be administered locally via aqueous eyedrop solution or local intra-lacrimal gland injection.

In still another aspect, the present invention comprises a composition for the treatment of a dry-eye disorder in a subject, the composition comprising a WNT signaling modulator. In any embodiment of the composition, the dry-eye disorder may be a Sjögren's syndrome disorder.

In an embodiment of the composition, the WNT signaling modulator may comprise at least one engineered bi-specific full-length IgG antibody that directly activates a canonical WNT signaling pathway. The at least one engineered bi-specific full-length IgG antibody may be specific for any one or more of Fzd1, Fzd,2, Fzd5, Fzd7, Fzd8, and Lrp6 or Lrp5.

In an embodiment, the composition may further comprise an at least one additional agent, including an anti-inflammatory agent, an artificial tear agent, or an lacrimal gland secretagogue. In particular, the anti-inflammatory agent may be antibiotics or steroids including cyclosporine A (e.g., Restasis®) and lifitegrast ophthalmic solution (e.g., Xiidra®). Artificial tears agent may include non-prescription eye drops that simulate tears, e.g., hydroxypropyl cellulose (Lacrisert®) inserts. Lacrimal gland secretagogue agents may include varenicline nasal spray, which selectively agonizes nicotinic acetylcholine receptors. Also, contemplated are autologous blood serum eye drops.

In any embodiment of the composition, the composition may comprise therapeutically effective amounts of each of its components.

In any embodiment of the composition, the subject may be a live mammal. For example, the subject may be mouse, rat, dog, cat, horse, or cow. In any embodiment of the composition, the subject may be a human patient.

In further embodiments, pharmaceutical compositions comprising an expression vector, e.g., a viral vector, comprising a polynucleotide comprising a nucleic acid sequence encoding a WNT antagonist/agonist molecule described herein and one or more pharmaceutically acceptable diluent, carrier, or excipient are also disclosed. In certain embodiments, the nucleic acid sequence encoding the WNT antagonist molecule and the nucleic acid sequence encoding the WNT agonist are in the same polynucleotide, e.g., expression cassette.

The present disclosure further contemplates a pharmaceutical composition comprising a cell comprising an expression vector comprising a polynucleotide comprising a promoter operatively linked to a nucleic acid encoding a WNT antagonist/agonist molecule and one or more pharmaceutically acceptable diluent, carrier, or excipient. In particular embodiments, the pharmaceutical composition further comprises a cell comprising an expression vector comprising a polynucleotide comprising a promoter operatively linked to a nucleic acid sequence encoding a WNT antagonist and a WNT agonist. In certain embodiments, the nucleic acid sequence encoding the WNT antagonist molecule and the nucleic acid sequence encoding the WNT agonist molecule are present in the same polynucleotide, e.g., expression cassette and/or in the same cell. In particular embodiments, the cell is a heterologous cell or an autologous cell obtained from the subject to be treated.

In particular embodiments, the cell is a stem cell, e.g., an adipose-derived stem cell or a hematopoietic stem cell. The present disclosure contemplates pharmaceutical compositions comprising a first molecule for delivery of a WNT antagonist molecule as a first active agent, and a WNT agonist as a second molecule. The first and second molecule may be the same type of molecule or different types of molecules. For example, in certain embodiments, the first and second molecule may each be independently selected from the following types of molecules: polypeptides, small organic molecules, nucleic acids encoding the first or second active agent (optionally DNA or mRNA, optionally modified RNA), vectors comprising a nucleic acid sequence encoding the first or second active agent (optionally expression vectors or viral vectors), and cells comprising a nucleic acid sequence encoding the first or second active agent (optionally an expression cassette).

The subject molecules, alone or in combination, can be combined with pharmaceutically acceptable carriers, diluents, excipients and reagents useful in preparing a formulation that is generally safe, non-toxic, and desirable, and includes excipients that are acceptable for mammalian, e.g., human or primate, use. Such excipients can be solid, liquid, semisolid, or, in the case of an aerosol composition, gaseous. Examples of such carriers, diluents and excipients include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile.

Pharmaceutical compositions may further include sterile aqueous solutions or dispersions and sterile powders for the extemporaneous preparation of sterile injectable solutions or dispersion. For intravenous administration, suitable carriers include physiological saline, bacteriostatic water, or phosphate buffered saline (PBS). In some cases, the composition is sterile and should be fluid such that it can be drawn into a syringe or delivered to a subject from a syringe. In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

Sterile solutions can be prepared by incorporating the WNT antagonist/agonist antibody or antigen-binding fragment thereof (or encoding polynucleotide or cell comprising the same) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

In one embodiment, the pharmaceutical compositions are prepared with carriers that will protect the antibody or antigen-binding fragment thereof against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions can also be used as pharmaceutically acceptable carriers. These can be prepared according to methods known to those skilled in the art.

It may be advantageous to formulate the pharmaceutical compositions in dosage unit form for ease of administration and uniformity of dosage. Dosage unit form as used herein refers to physically discrete units suited as unitary dosages for the subject to be treated; each unit containing a predetermined quantity of active antibody or antigen-binding fragment thereof calculated to produce the desired therapeutic effect in association with the required pharmaceutical carrier. The specification for the dosage unit forms is dictated by and directly dependent on the unique characteristics of the antibody or antigen-binding fragment thereof and the particular therapeutic effect to be achieved, and the limitations inherent in the art of compounding such an active antibody or antigen-binding fragment thereof for the treatment of individuals.

The pharmaceutical compositions can be included in a container, pack, or dispenser, e.g., eye dropper, e.g., a prefilled eye dropper, together with instructions for administration.

The pharmaceutical compositions of the present disclosure may be delivered to a subject in the form of a pill, capsule, cream, salve, syrup, dermal patch, suppository, intravenous drip, local injection aqueous solution, non-aqueous solution, eye wash solution, or any combination of thereof.

The pharmaceutical compositions of the present disclosure may be delivered to a subject by direct ophthalmic application, muscular injection, intra-lacrimal gland injection, subconjunctival injection, meibomian gland injection, intravenous injection, peritoneal injection, nasally, orally, rectally, or any combination thereof.

The pharmaceutical compositions of the present disclosure encompass any pharmaceutically acceptable salts, esters, or salts of such esters, or any other compound which, upon administration to an animal comprising a human, is capable of providing (directly or indirectly) the biologically active antibody or antigen-binding fragment thereof.

The present disclosure includes pharmaceutically acceptable salts of a WNT antagonist/agonist molecule described herein. The term “pharmaceutically acceptable salt” refers to physiologically and pharmaceutically acceptable salts of the compounds of the present disclosure: i.e., salts that retain the desired biological activity of the parent compound and do not impart undesired toxicological effects thereto. A variety of pharmaceutically acceptable salts are known in the art and described, e.g., in “Remington's Pharmaceutical Sciences”, 17th edition, Alfonso R. Gennaro (Ed.), Mark Publishing Company, Easton, PA, USA, 1985 (and more recent editions thereof), in the “Encyclopedia of Pharmaceutical Technology”, 3rd edition, James Swarbrick (Ed.), Informa Healthcare USA (Inc.), NY, USA, 2007, and in J. Pharm. Sci. 66:2 (1977). Also, for a review on suitable salts, see “Handbook of Pharmaceutical Salts: Properties, Selection, and Use” by Stahl and Wermuth (Wiley-VCH, 2002). Pharmaceutically acceptable base addition salts are formed with metals or amines, such as alkali and alkaline earth metals or organic amines.

Metals used as cations comprise sodium, potassium, magnesium, calcium, and the like. Amines comprise N—N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure.

In some embodiments, the pharmaceutical composition provided herein comprise a therapeutically effective amount of a WNT antagonist/agonist molecule or pharmaceutically acceptable salt thereof in admixture with a pharmaceutically acceptable carrier, diluent and/or excipient, for example saline, phosphate buffered saline, phosphate and amino acids, polymers, polyols, sugar, buffers, preservatives and other proteins. Exemplary amino acids, polymers and sugars and the like are octylphenoxy polyethoxy ethanol compounds, polyethylene glycol monostearate compounds, polyoxyethylene sorbitan fatty acid esters, sucrose, fructose, dextrose, maltose, glucose, mannitol, dextran, sorbitol, inositol, galactitol, xylitol, lactose, trehalose, bovine or human serum albumin, citrate, acetate, Ringer's and Hank's solutions, cysteine, arginine, carnitine, alanine, glycine, lysine, valine, leucine, polyvinylpyrrolidone, polyethylene and glycol. Preferably, this formulation is stable for at least six months at 4° C.

In some embodiments, the pharmaceutical composition provided herein comprises a buffer, such as phosphate buffered saline (PBS) or sodium phosphate/sodium sulfate, tris buffer, glycine buffer, sterile water and other buffers known to the ordinarily skilled artisan such as those described by Good et al. (1966) Biochemistry 5:467. The pH of the buffer may be in the range of 6.5 to 7.75, preferably 7 to 7.5, and most preferably 7.2 to 7.4.

From the foregoing it will be appreciated that, although specific embodiments of the present disclosure have been described herein for purposes of illustration, various modifications may be made without deviating from the spirit and scope of the present disclosure. Accordingly, the present disclosure is not limited except as by the appended claims.

The broad scope of this invention is best understood with reference to the following example, which is not intended to limit the inventions to the specific embodiments.

### Examples

### I. Methods

Standard methods in molecular biology were used, including methods described, e.g., in Maniatis et al. (1982) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif Standard methods also appear in Ausbel et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described, e.g., in Coligan et al. (2000) Current Protocols in Protein Science, Vol. 1, John Wiley and Sons, Inc., New York. Chemical analysis, chemical modification, post-translational modification, production of fusion proteins, glycosylation of proteins are described in, e.g., Coligan et al. (2000) Current Protocols in Protein Science, Vol. 2, John Wiley and Sons, Inc., New York; Ausubel et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391. Production, purification, and fragmentation of polyclonal and monoclonal antibodies are described, e.g., in Coligan et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra. Standard techniques for characterizing ligand/receptor interactions are available in, e.g., Coligan et al. (2001) Current Protocols in Immunology, Vol. 4, John Wiley, Inc., New York.

Methods for flow cytometry, including fluorescence activated cell sorting detection systems (FACS®), are available in, e.g., Owens et al. (1994) Flow Cytometry Principles for Clinical Laboratory Practice, John Wiley and Sons, Hoboken, N.J.; Givan (2001) Flow Cytometry, 2^{nd} ed.; Wiley-Liss, Hoboken, N.J.; Shapiro (2003) Practical Flow Cytometry, John Wiley and Sons, Hoboken, N.J. Fluorescent reagents suitable for modifying nucleic acids, including nucleic acid primers and probes, polypeptides, and antibodies, for use, e.g., as diagnostic reagents, are available In, e.g., Molecular Probes (2003) Catalogue, Molecular Probes, Inc., Eugene, Oreg.; Sigma-Aldrich (2003) Catalogue, St. Louis, Mo.

Standard methods of histology of the immune system are described in, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.

Software packages and databases for determining, e.g., antigenic fragments, leader sequences, protein folding, functional domains, glycosylation sites, and sequence alignments, are available, in, e.g., GenBank, Vector NTI® Suite (Informax, Inc, Bethesda, Md.); GCG Wisconsin Package (Accelrys, Inc., San Diego, Calif); DeCypher® (TimeLogic Corp., Crystal Bay, Nev.); Menne et al. (2000) Bioinformatics 16: 741-742; Menne et al. (2000) Bioinformatics Applications Note 16:741-742; Wren et al. (2002) Comput. Methods Programs Biomed. 68:177-181; von Heijne (1983) Eur. J. Biochem. 133:17-21; von Heijne (1986) Nucleic Acids Res. 14:4683-4690.

### II. Organoid Establishment and Expansion

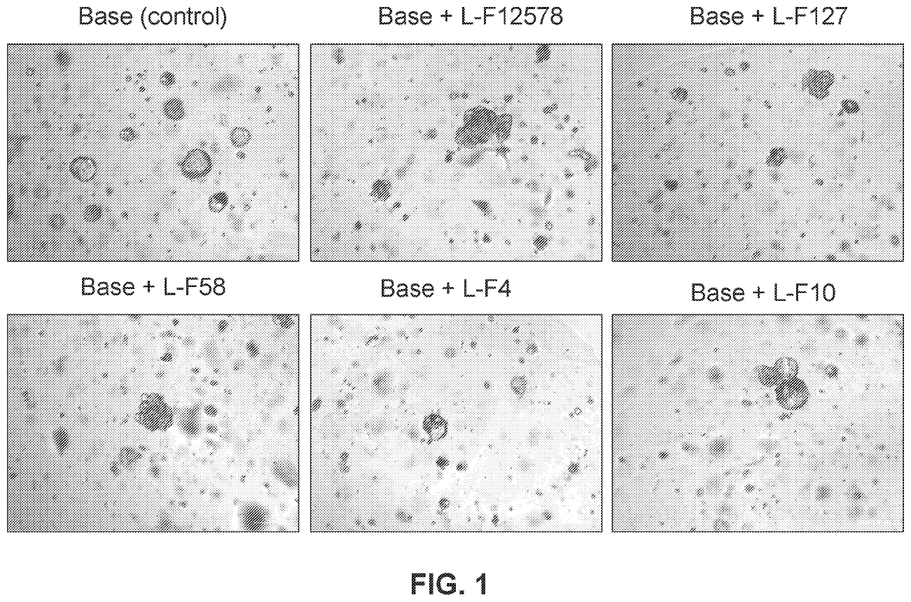

Lacrimal glands and salivary glands from wildtype C57BL/6 mice were dissected and stored in Advanced DMEM/F12 (GIBCO) on ice. Multiple murine lacrimal glands or salivary glands were pooled in a petri dish and muscle, ducts and connective tissue were removed and discarded as much as possible. The remaining glandular epithelium was chopped into approximately 1 mm pieces using a scalpel. The tissue pieces were enzymatically digested in collagenase (Sigma-Aldrich, C9407, 1 mg/mL) solution containing 10 μM ROCK inhibitor Y-27632 (Abmole, M1817) in Advanced DMEM/F12 (GIBCO) for about 15 minutes shaking at 37° C. The homogeneous cell suspension was pelleted (1200 rpm for 5 minutes) and washed twice with Advanced DMEM/F12 prior to plating. The same protocol was applied to human post-mortem lacrimal gland material. Cells were plated in 20 L droplets of Cultrex Pathclear Reduced Growth Factor Basement Membrane Extract (BME) (3533-001, Amsbio) or Matrigel™ GFR Membrane Matrix (Corning CB40230C). After a 15-minute solidification, expansion medium containing various mammalian growth factors and cytokines, RSPO1, and a surrogate WNT (L-F12578; L-F127; L-F58; L-F4; or L-F10 at 5 nM) was added. As shown in FIG. 1, cells treated with L-F12578, L-F127 or L-F58 expanded rapidly with a solid budding morphology, whereas cells treated with the control and other surrogate WNT treatments had little growth or a ductal morphology (see, e.g., Bannier-Helaouet, et al. (2021) Cell Stem Cell 28:1221-1232).

### III. Surrogate WNT Screening in Acinar Cells

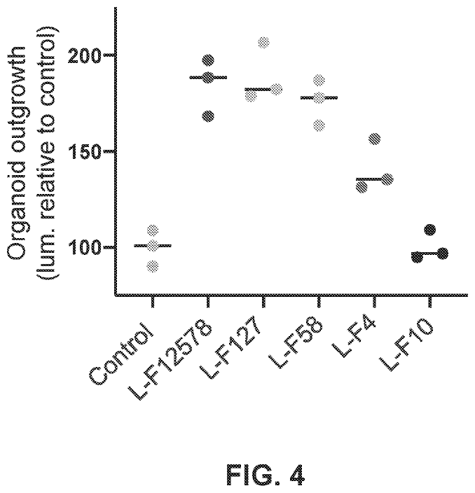

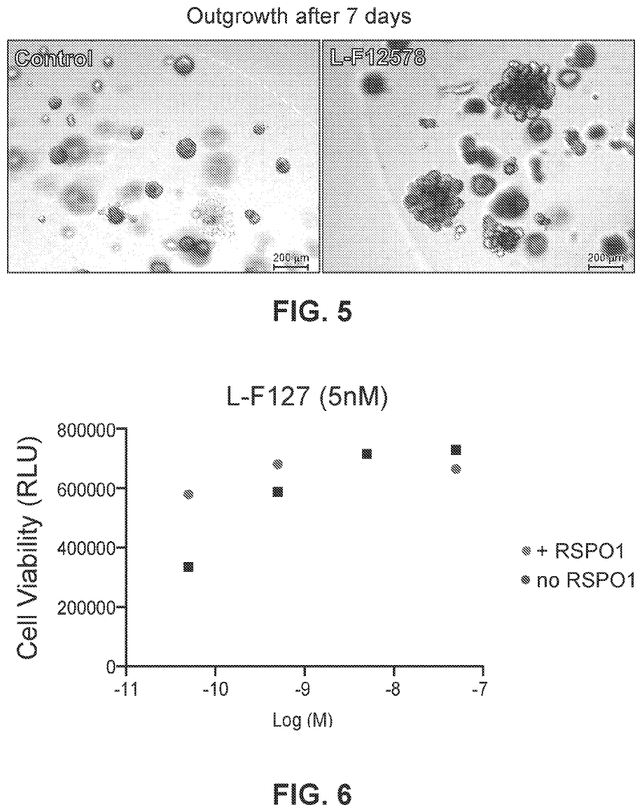

For outgrowth activity screening of surrogate WNTs, i.e., WNT mimetics, mouse lacrimal gland or salivary gland primary cells or organoid cells (<passage 5) were digested into a near single-cell suspension using TrypLE (Thermo Fisher) for 10 minutes at 37° C. The base medium for activity assays consisted of expansion medium without RSPO1 or any surrogate WNT and supplemented with 1 M porcupine inhibitor Wnt-C59 (#5148 Tocris) and 10 M Y-27632 (#5092280001 MilliporeSigma). Experimental conditions consisted of one or a combination of 500 ng/mL recombinant RSPO1 and 5 nM surrogate WNT (L-F12578, L-F127, L-F58, L-F4, or L-F10), unless otherwise stated. All cells for all conditions were plated in 15 μL Matrigel droplets in 96-well plates and submerged in 120 μL of the experimental medium in round bottom 96-well plate. Cells were grown out as organoids for 7 days prior to quantification. Outgrowth efficiency was quantified using cell viability assay CellTiter-Glo® (G9683 Promega), measured on the SpectraMax Paradigm microplate reader (Molecular Devices) according to manufactures protocols, and the results are shown in FIGS. 4 and 5. For RSPO dependency assay, organoids were cultures with a dose-range of surrogate WNT (0.05, 0.5, 5, 50 nM) with or without 500 ng/mL RSPO1, and the results are shown in FIG. 6.

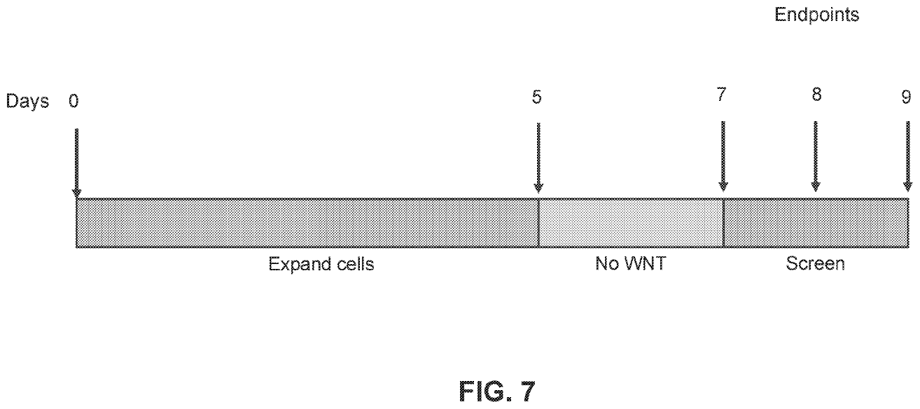

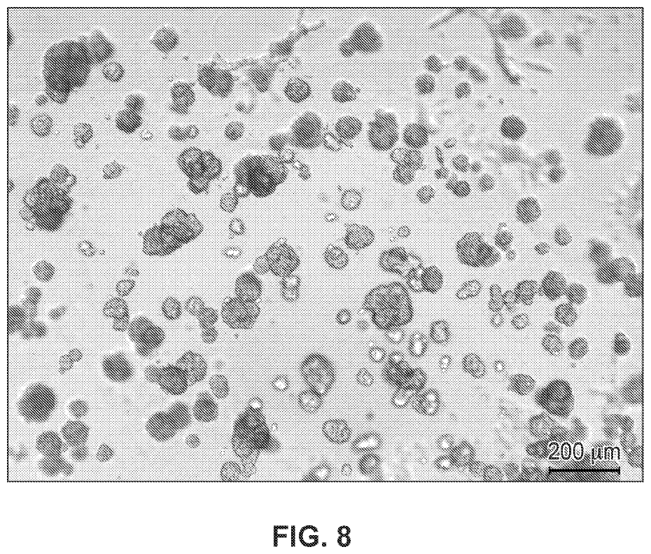

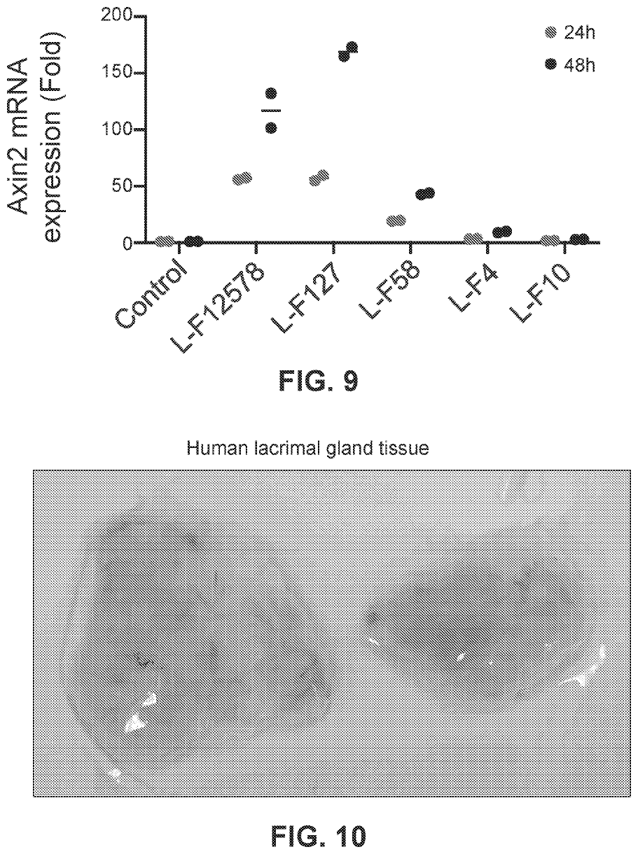

For WNT target gene expression screening upon surrogate WNT stimulation, mouse acinar cell organoids were grown in complete expansion days for 5 days prior to withdrawal of RSPO1, surrogate WNT and addition of 1 M porcupine inhibitor Wnt-C59 (#5148 Tocris) for 48 hours, as shown in FIG. 7. After the WNT withdrawal period, RSPO1 and/or 5 nM surrogate WNT (L-F12578, L-F127, L-F58, L-F4, or L-F10) were reintroduced for the indicated time period to assess WNT target gene induction, as shown in FIG. 8. RNA was extracted using the QIAprep miniprep kit (#27104 QIAGEN) according to manufacturer's protocol. Expression of Axin2 was determined by qPCR using SYBR Green (#K0243 Thermo Scientific) according to manufacturer's protocol, and the results are shown in FIG. 9.

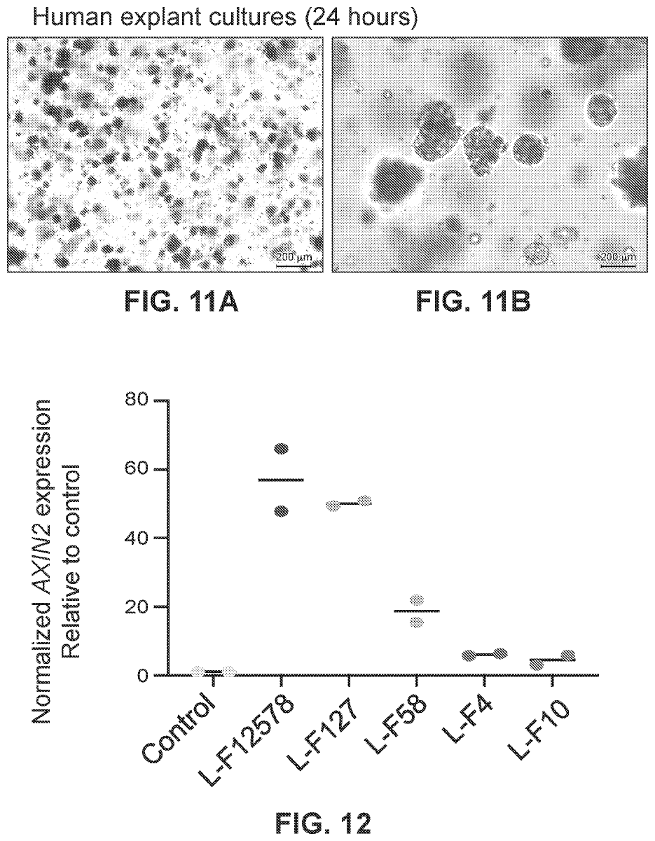

Human AXIN2 induction in Surrogate WNT screen was performed in human lacrimal gland explant cultures, derived from fresh human lacrimal gland tissue (shown in FIG. 10), in complete expansion medium. Cells were plated in Matrigel as described above (see FIGS. 11A-11B), and exposed to different surrogate WNTs (L-F12578, L-F127, L-F58, L-F4, or L-F10) in combination with RSPO1 from the moment of plating. After 24-hour exposure, the RNA was extracted using the QIAGEN miniprep kit according to manufacturer's protocol. Expression of AXIN2 was determined by qPCR using SYBR Green (Thermo Fisher) according to manufacturer's protocol (primers sequences provided below), and the results are shown in FIG. 12.

### IV. Immunofluorescence Staining and In Situ Hybridization

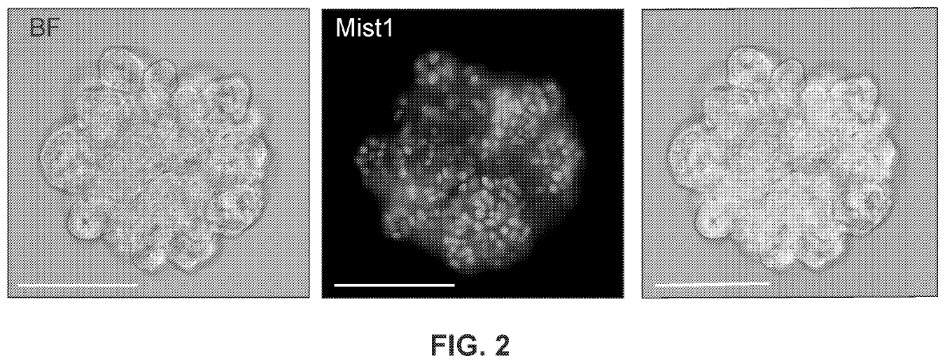

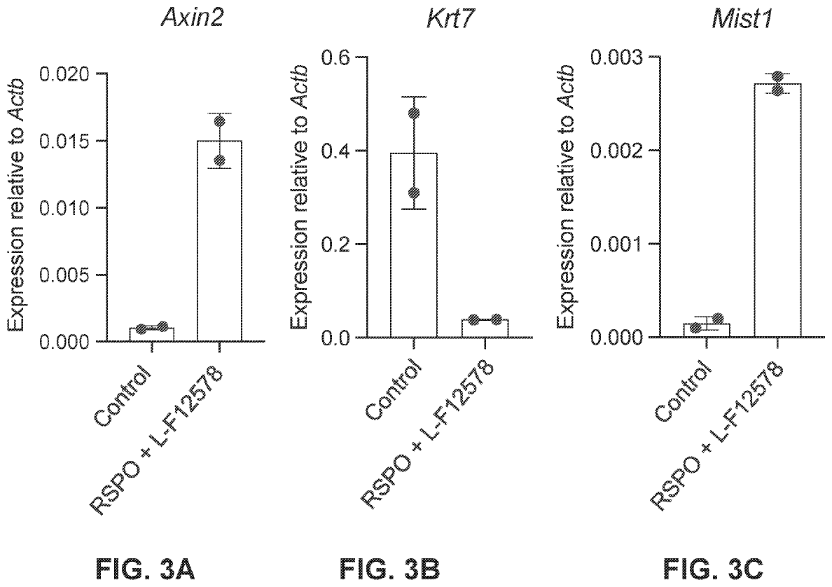

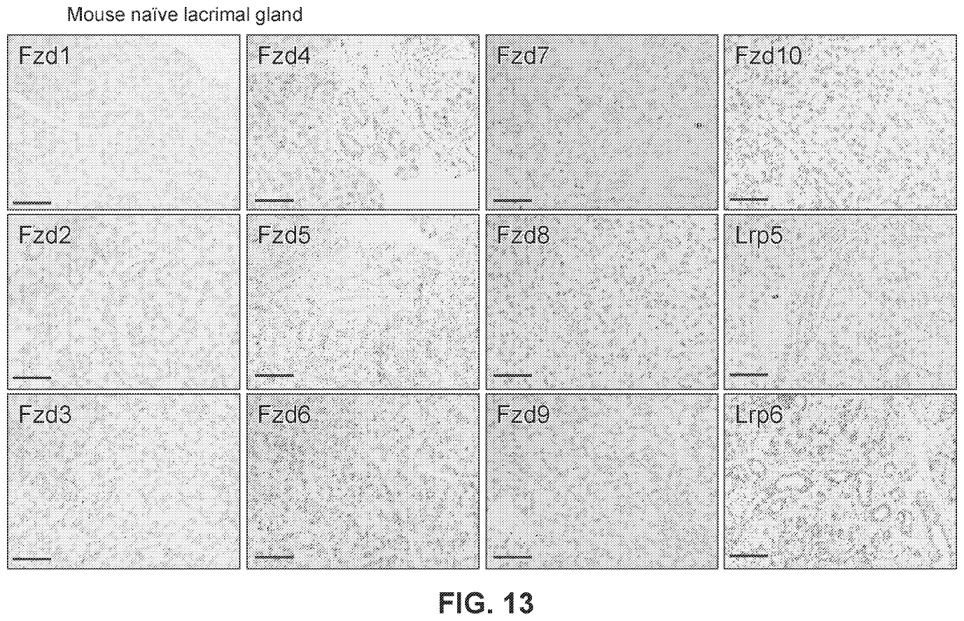

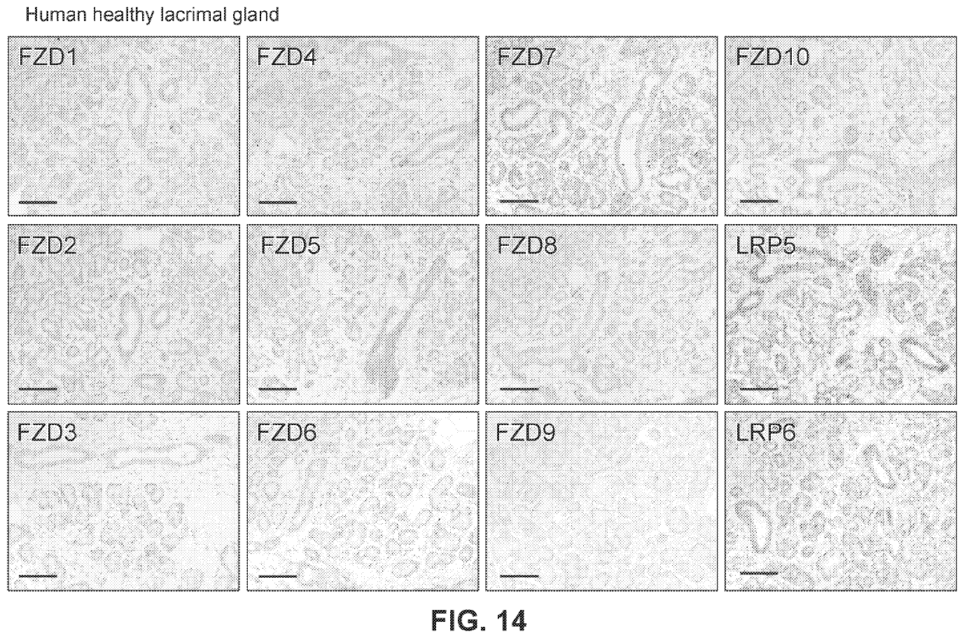

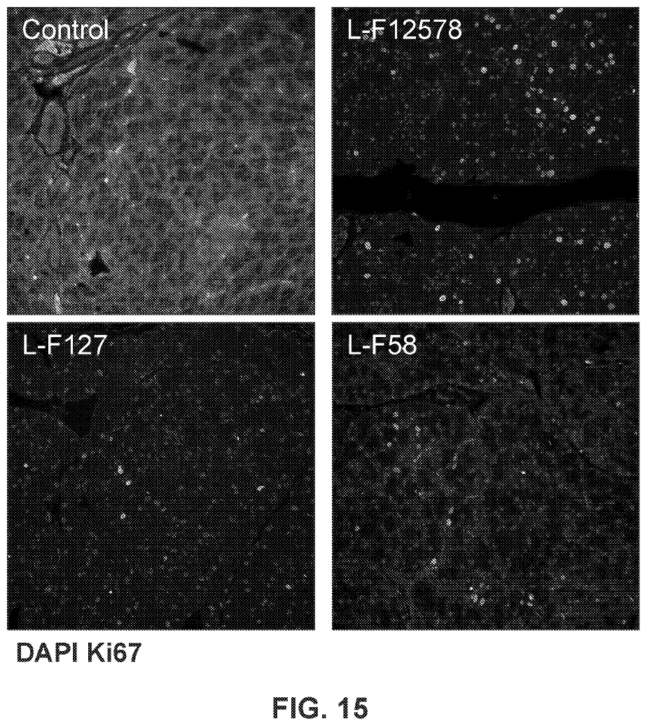

Organoids were harvested in cell recovery solution (354253, Corning) and fixed in 4% formaldehyde (R37814 Sigma-Aldrich) for at least 2 hours at room temperature and permeabilized using 0.2% Triton X-100 in PBS (#ICN19485450 Fisher Scientific). Whole-mount staining was performed overnight in 2% donkey serum using rabbit anti-MIST1 (#14896 CST) and DAPI (#EN62248 Fisher Scientific), secondary antibody was Alexa Fluor 568 donkey anti-rabbit IgG (Life Technologies, A10042). Organoids were imaged on a Leica THUNDER imaging system (FIG. 2). Immunofluorescence staining of murine lacrimal glands was done on sections from paraffin embedded tissue. In short, sections were deparaffinized, heat antigen retrieval, permeabilized, blocked, and stained for Ki67 (#ab15580 Abcam) and DAPI (#EN62248 Fisher Scientific). Secondary staining was performed using the secondary antibody, Alexa Fluor 568 donkey anti-rabbit IgG (Life Technologies, A10042), and imaging was performed on a Leica DMi8 system (FIG. 15). In situ hybridizations were performed according to manufactures protocol (ACDBio) with human or mouse specific probes for indicated WNT receptors and imaged on a Leica DMi8 system. The results are shown in FIGS. 13-14.

### V. Animals and Treatment

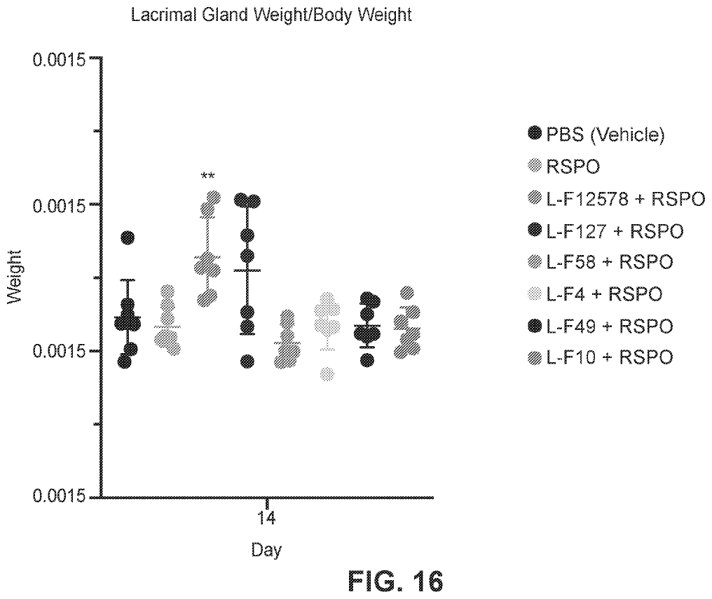

Female C57BL/6 mice (8-10-week-old) were intraperitoneally dosed on day 0, 3, 7 and 10 at 10 mpk with various Wnt mimetics. At day 14, proliferating cells were detected in L-F12578, L-F127 and L-F58 treatment groups by Ki67 signal (FIG. 15). A similar timing of dosing with WNT mimetics in combination with RSPO resulted in a lacrimal gland weight increase in the L-F12578 and L-F127 treatment groups after two weeks compared to control and RSPO alone (FIG. 16).

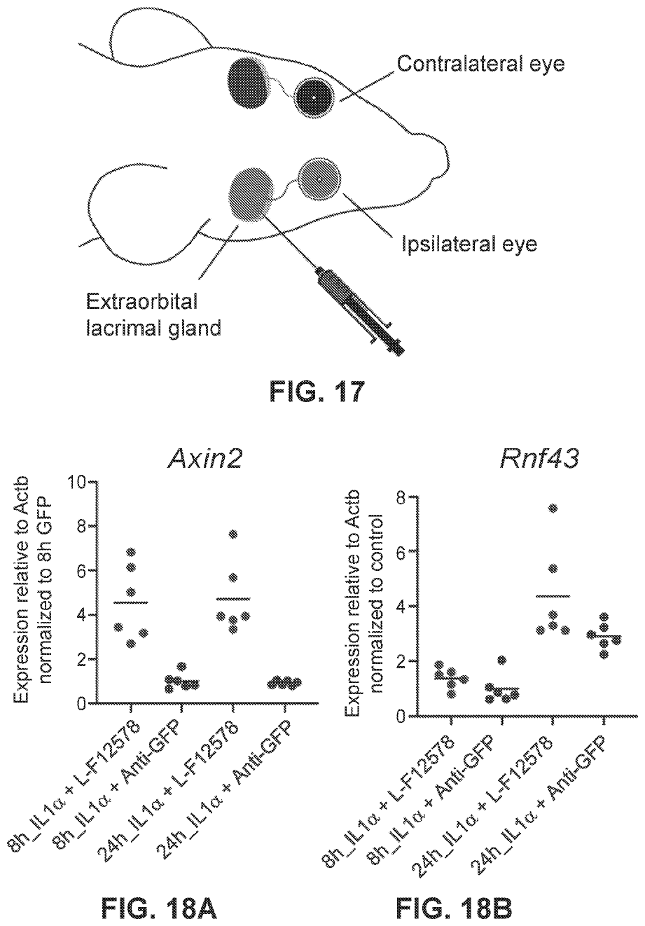

Female C57BL/6 mice (8-10-week-old) were purchased from Jackson Laboratory (Bar Harbor, ME). All animal procedures were performed in accordance with IACUC committee's regulations. Mice were anesthetized and the left extraorbital lacrimal glands were left untreated while the right lacrimal glands were injected with IL-1α or a cocktail of IL-1α and SWAP™, i.e., WNT mimetic, as shown in FIG. 17.

All animal experiments were performed according to national ethical guidelines in addition to the guidance and approval by the Institutional Animal Care and Use Committee (IACUC) of Surrozen, Inc. Twelve-week-old MRL-lpr (stock 000485) female mice were obtained from Jackson Laboratories (Bar Harbor, ME, USA) and were housed 4 per cage. Proteins treatments at 3 mg per kg and 10 mg per kg were intraperitoneally dosed on day 0, 3, 7 and 10 in C57Bl/6J mice and MRL-lpr along with MRL/MpJ mice respectively. RSPO2-nFcFc treatment at 0.1, 0.3, 1 and 3 mg per kg were intraperitoneally dose on day 0, 3, 7 and 10 in C57Bl/6J mice. Bone mineral density (BMD) and fat content of animals were measured via in vivo DEXA method using a Faxitron UltraFocus (Faxitron Bioptics, Tucson, Arizona) on day 0, 7 and 13. Animals were anesthetized during imaging through isoflurane and sample ROI included the entire murine skeleton except material above the cervical spine due to increased radiographical intensity of the skull. BMD and fat content were calculated using the accompanying Vision DXA software. Animals were terminated on day 14, and salivary glands were collected for histology.

### VI. Intra-Lacrimal Gland Injection

Intra-lacrimal gland injection was performed as previously described with minor alteration in Zoukhri et al. A Single Injection of Interleukin-1 Induces a Reversible Aqueous-tear Deficiency, Lacrimal Gland Inflammation, and Acinar and Ductal Cell Proliferation, 84 EXP. EYE RES. 894-904 (2007). Briefly, a small incision anterior to the ear of isoflurane-anesthetized animal was made to expose the extraorbital lacrimal gland. A volume of 1 μL was injected 3 times (total volume of 3 μL) to the exposed lacrimal gland.

The following procedure was established for lacrimal gland duct ligation, adapted from Liu et al. 2017. Animals are anesthetized with isoflurane. After the mouse is no longer responsive to pinches on two extremities in two different body quadrants, it is moved to the stage of a dissecting scope and fashioned with a nosecone to continue anesthesia by isoflurane. A small incision anterior to the ear is made to expose the extraorbital lacrimal gland which is lifted to expose the main excretory duct. A size 4-0 silk thread is then used to ligate the duct. After duct ligation, the skin incision is closed, sutured, and antibiotic ointment is then applied to the skin. The contralateral lacrimal gland is not operated on and served as a control. After three days, the skin suture is reopened to expose the lacrimal gland. The duct ligation is then released by cutting the silk suture with micro scissors. In the same surgery, treatment groups are injected with 2 mL of test article into the lacrimal gland ipsilateral to the ligation. After injection, the skin incision is closed, sutured, and antibiotic ointment is then applied. The animals are then sacrificed, and ipsilateral and contralateral lacrimal glands collected 24 h, 3 days or 14 days after releasing the ligation.

### VII. Induction of Dry Eye and SWAP™ Treatment

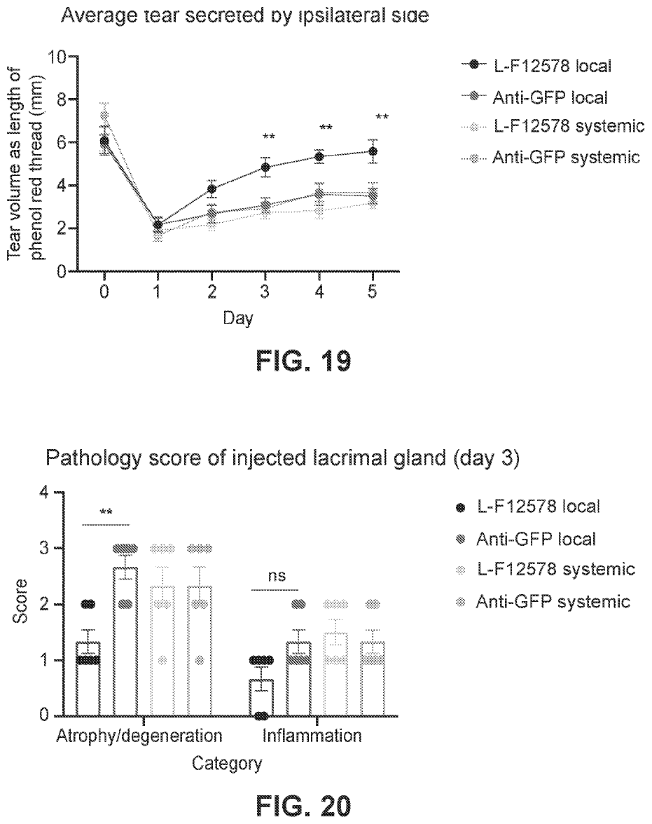

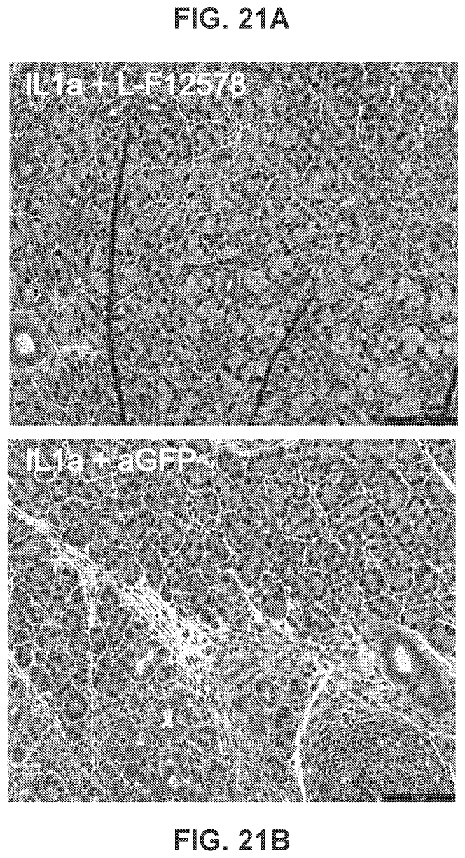

For induction of dry eye, 3 μL of recombinant human IL-1α (Peprotech) at 2 g/L was injected to the lacrimal gland. For local SWAP™ treatment, SWAP™ (3.5 μg/μL) was mixed with IL-1α (2 μg/μL) in a total volume of 3 μL then injected to the lacrimal gland. For systemic SWAP™ treatment, SWAP™ was intraperitoneally injected at 10 mg/kg twice a week for 2 weeks, as shown in FIGS. 19-20.

### VIII. Measurement of Aqueous Tear Secretion

Aqueous tear secretion was measured using phenol-impregnated cotton threads (Zone-Quick, Menicon) on restrained, unanesthetized mice. The threads were held with forceps and applied to the lateral canthus of both eyes for 30 seconds. Wetting of the thread which turned from yellow to red in contact with tears was measured in millimeters, and the results are shown in FIG. 19.

### IX. WNT Mimetic Molecules Induce Salivary Gland Hypertrophy

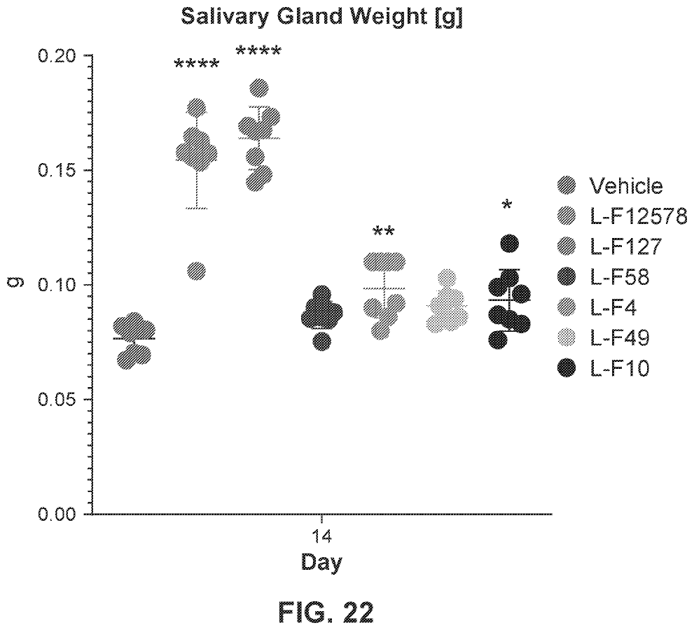

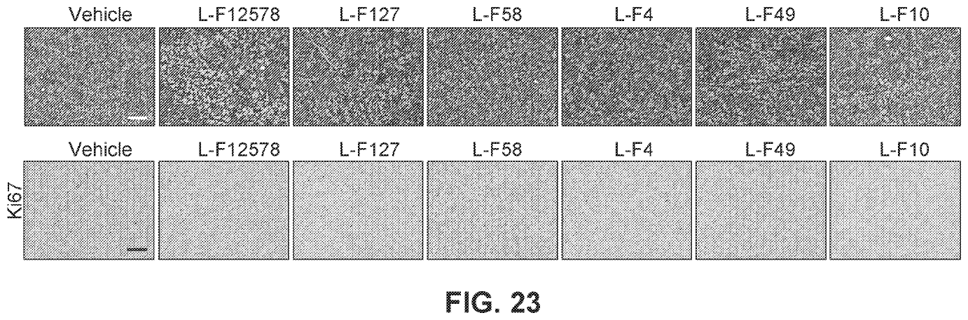

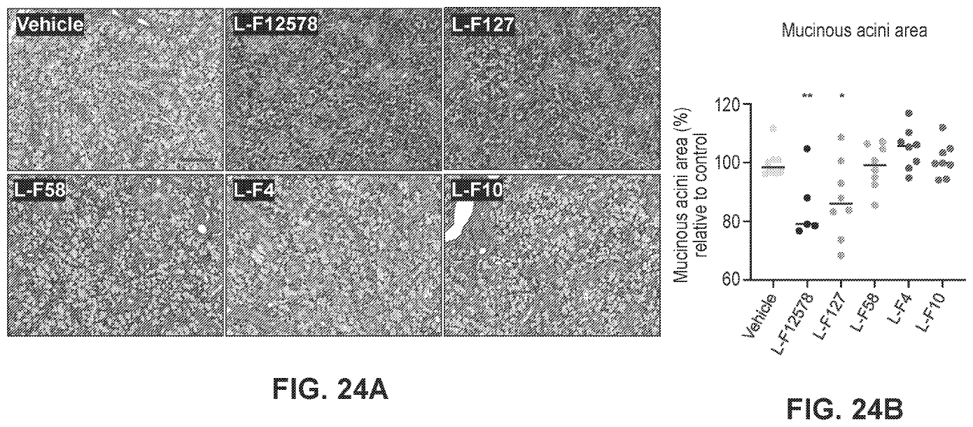

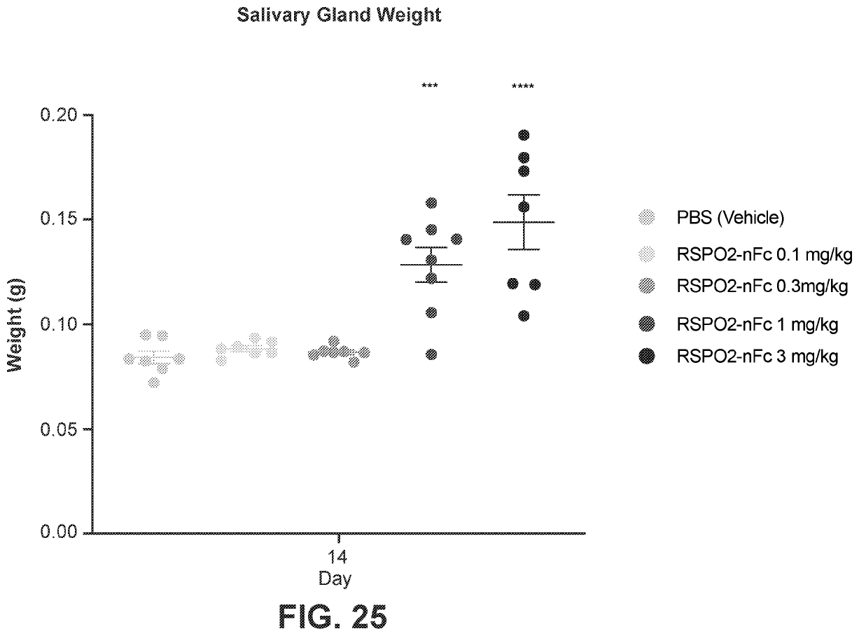

The effect of systemic WNT mimetic administration on salivary gland weight and histology in vivo, was examined. Significant increases in salivary gland weight in the groups of L-F12578, L-F127, L-F4, and L-F10 dosed at 3 mpk compared with Vehicle group on day 14, by 101% (P<0.001), 114% (P<0.001), 29% (p<0.01) and 22% (P<0.05), respectively, were observed, with the effects of L-F12578 and L-F127 being the most pronounced (FIG. 22). None of the WNT mimetic molecules, targeting different FZD receptors, had any observable effect on the histology of the submandibular gland in vivo by H&E stain (FIG. 23A). Staining for proliferative cells by Ki67 showed little difference between treatment groups, indicating a potential earlier peak of cell division driving the differences in organ weight (FIG. 23B). Animals dosed with the same molecules but at 10 mpk (for 14 days, bi-weekly systemic treatment) did have a clear histopathology phenotype. L-F12578 and L-F127 treated animals displayed a difference in staining in the seromucinous glandular acini with an increase in the amount of cytoplasmic basophilia determined by an independent pathologist (FIG. 24A). Automated quantification of the mucinous and serous portion of the salivary gland by Image J shows a significant reduction in the mucinous acini area in L-F12578 and L-127 treated animals (FIG. 24B). Also, systemic RSPO2 administration elicited a hypertrophic response in the salivary gland. In a two-week treatment with a range of RSPO2-nFc doses the 1 mpk and 3 mpk dose have a significant increase in salivary gland weight at day 14 (FIG. 25).

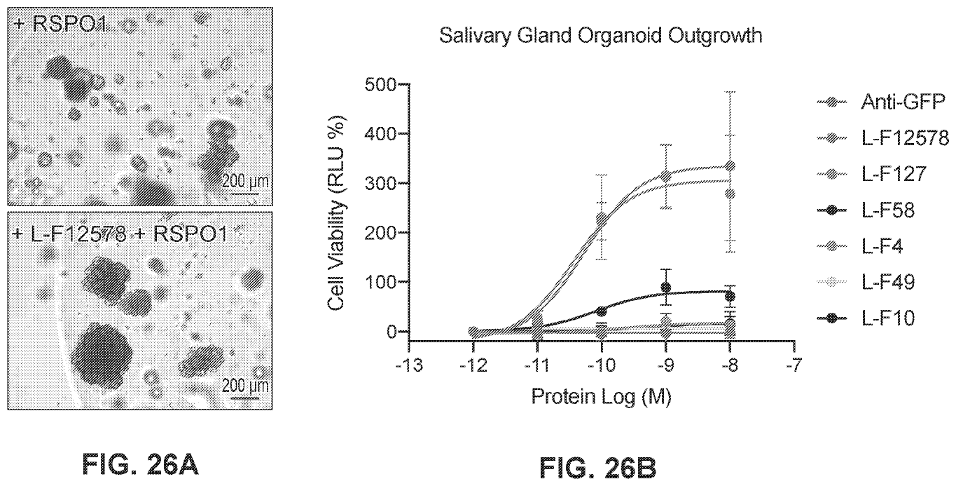

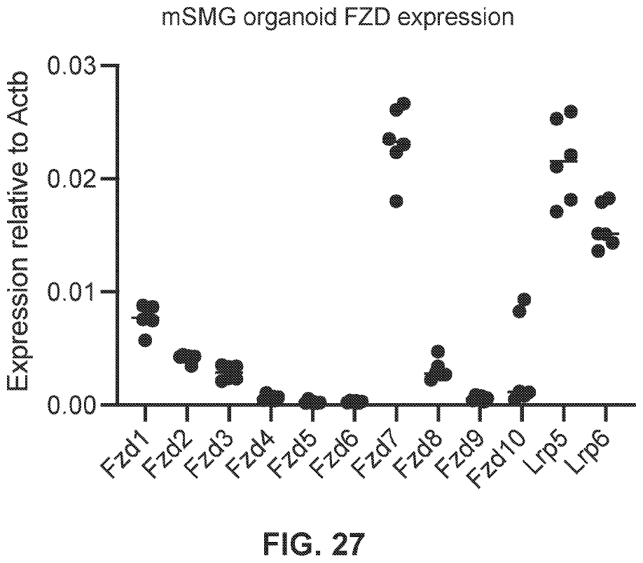

To understand the effect of WNT activation on salivary gland cells, murine salivary gland organoids as an experimental platform (see, e.g., Maimets et al. (2016) Stem Cell 6:150-162) were utilized. Treatment with L-F12578 (5 nM) in combination with recombinant RSPO1 increased proliferation compared to RSPO1 alone, resulting in the fast expansion of larger organoids (FIG. 26A). FZD specificity of this pro-proliferation phenotype was examined by screening the WNT mimetics in an outgrowth assay and compared it to anti-GFP antibody. In a dose-dependent manner, L-F12758 and L-F127 significantly increased the outgrowth efficiency measured as cell viability at day 10, and a modest effect was observed for L-F58 at high concentrations (FIG. 26B). Fzd7, Fzd1 and Fzd2 were shown to be the highest expressed Frizzled's in murine salivary gland organoids consistent with the in vitro and in vivo effects on salivary gland (FIG. 27).

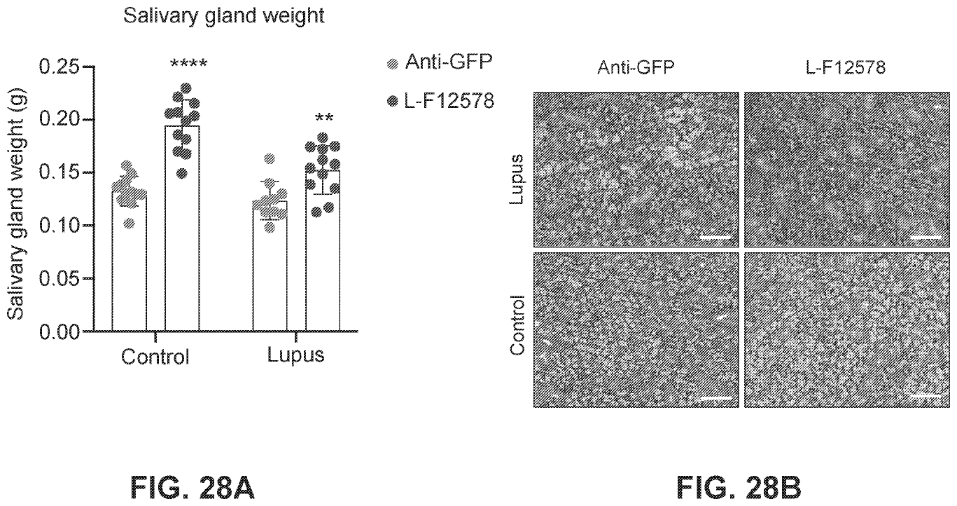

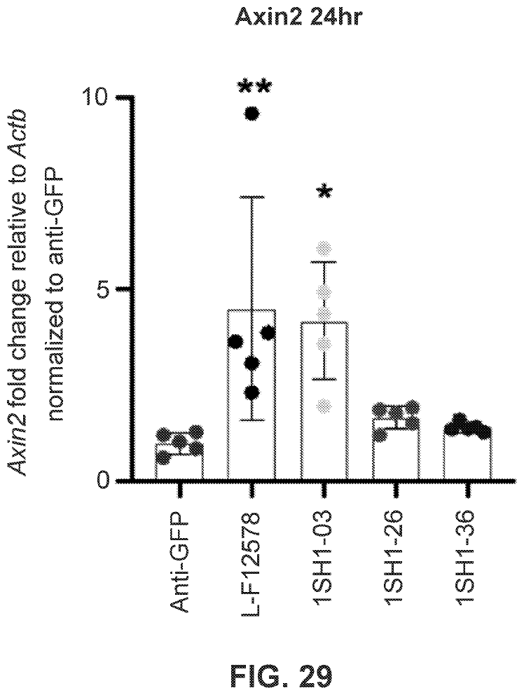

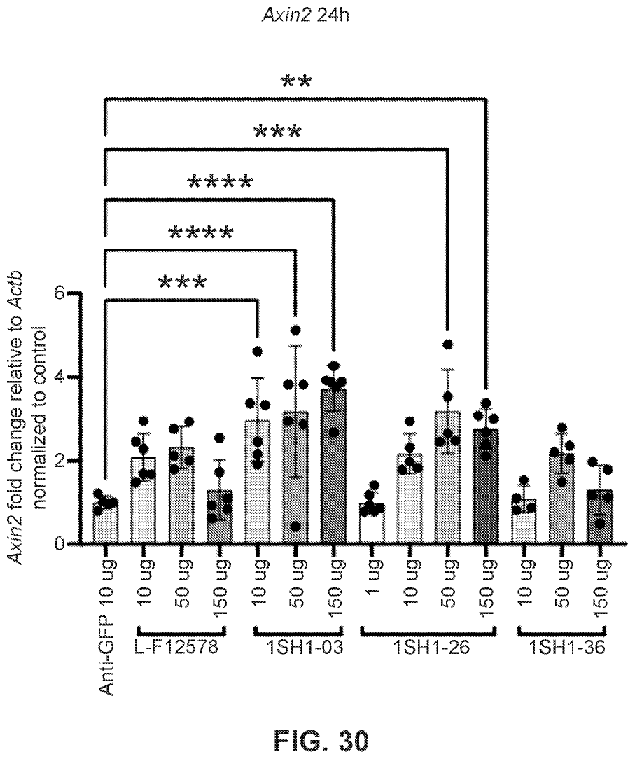

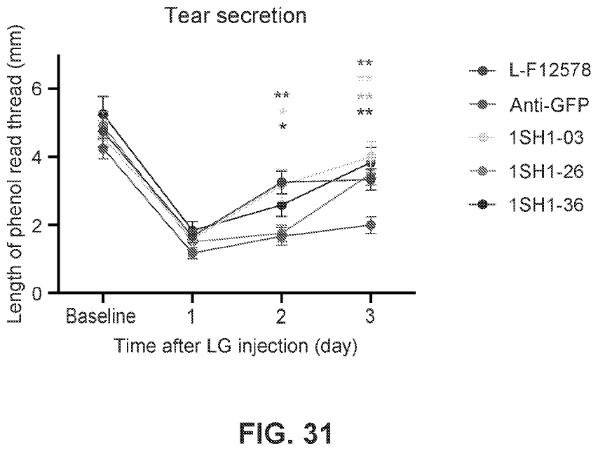

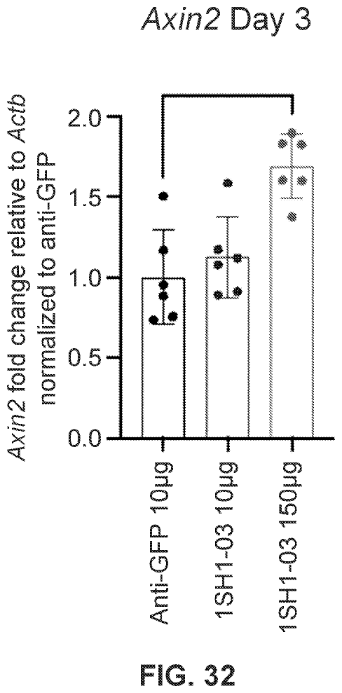

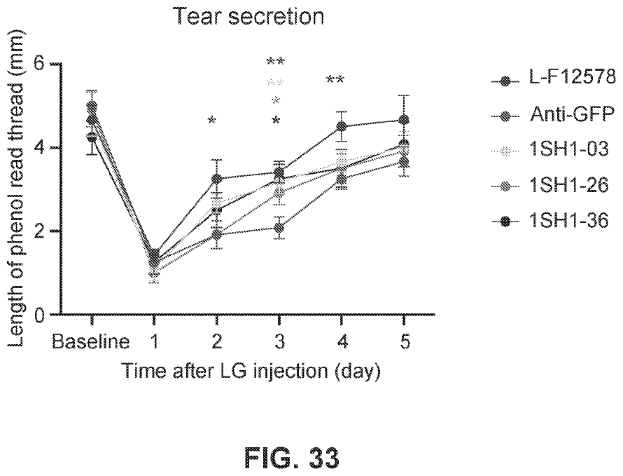

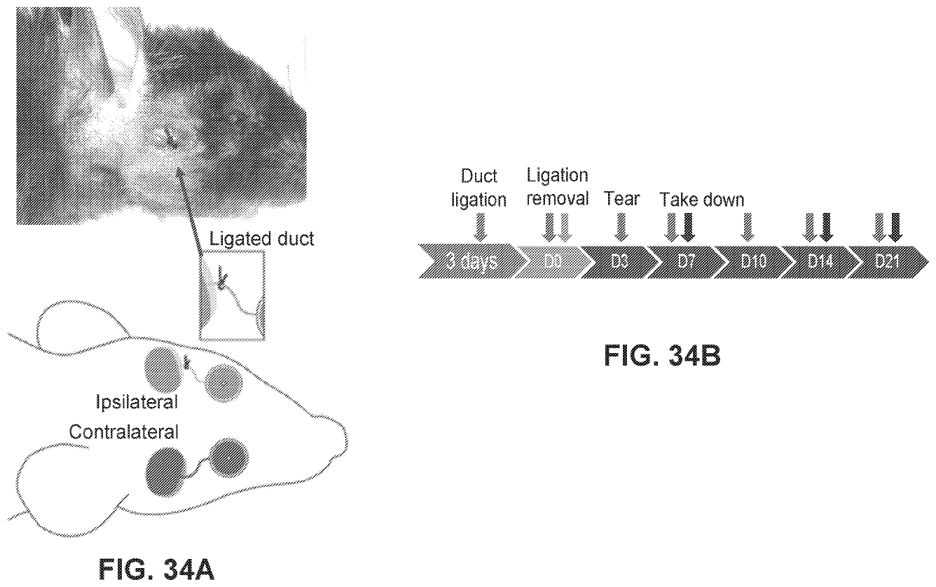

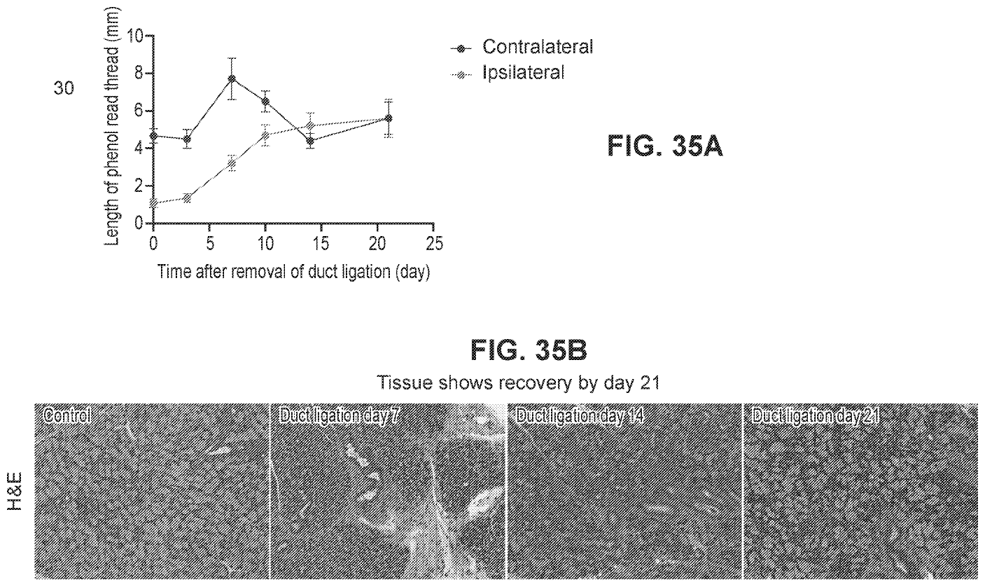

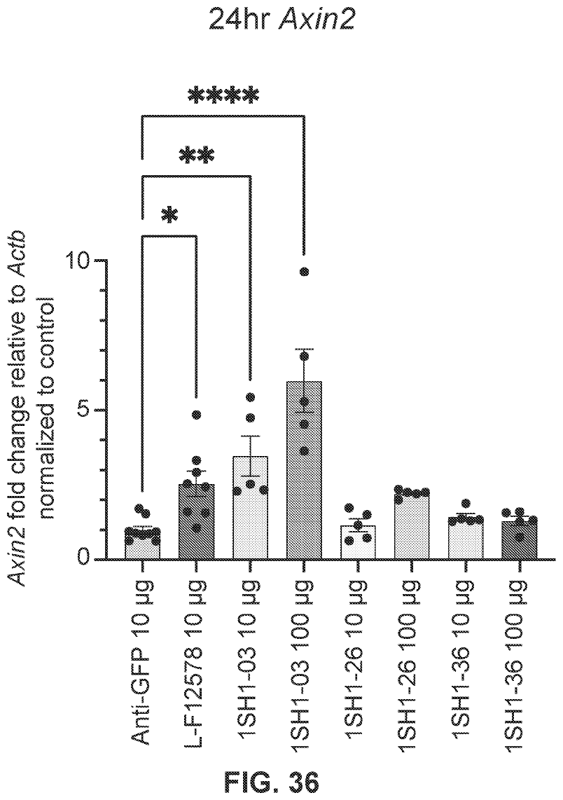

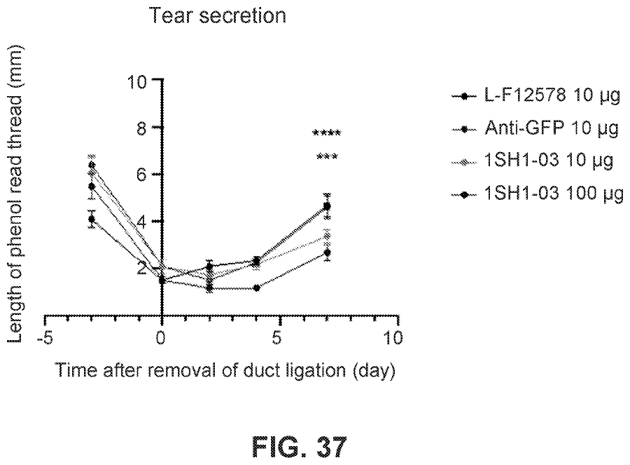

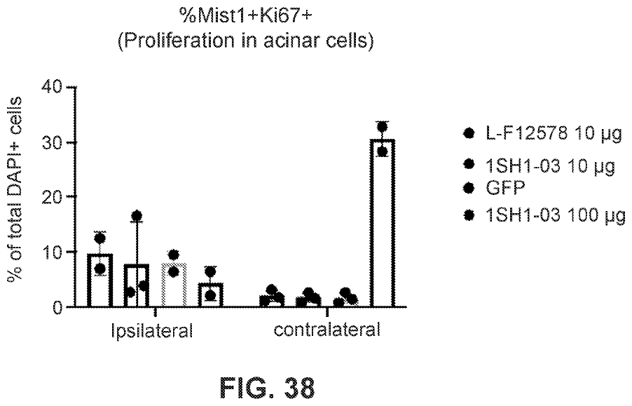

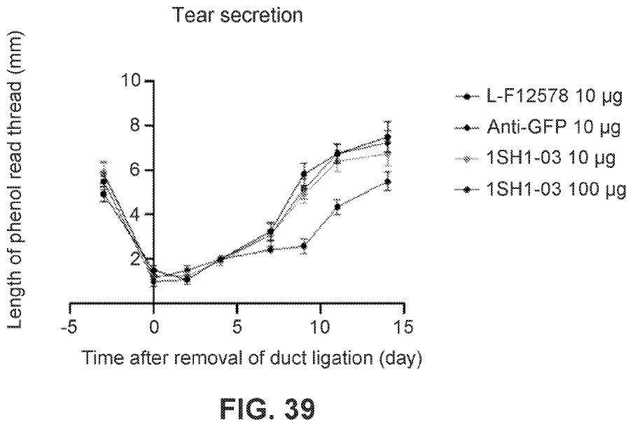

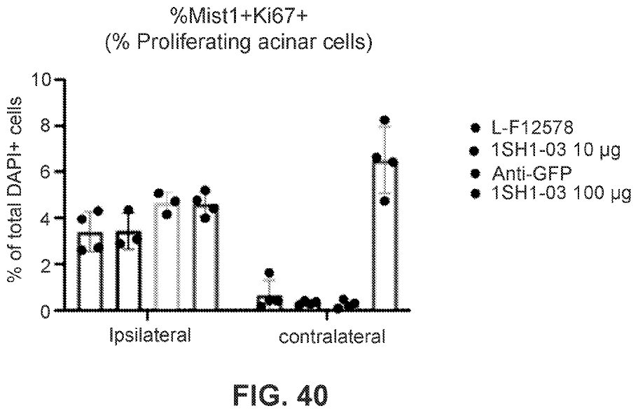

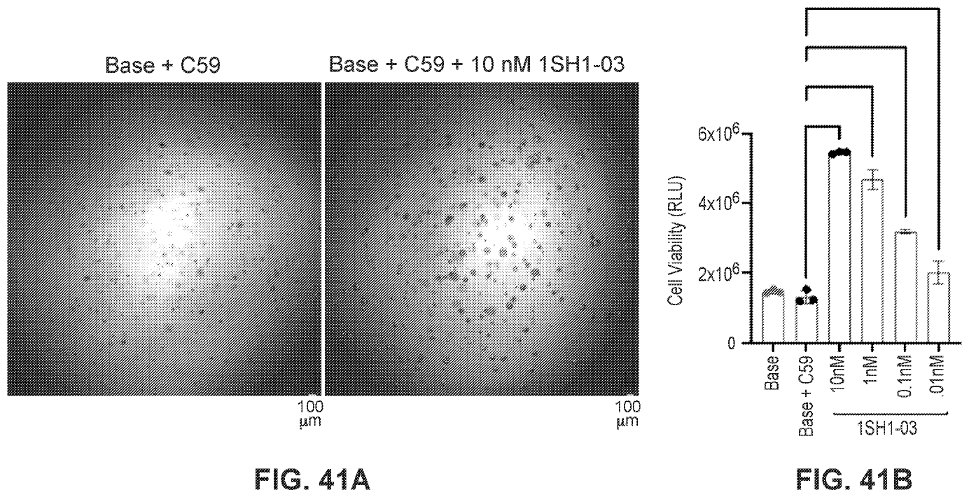

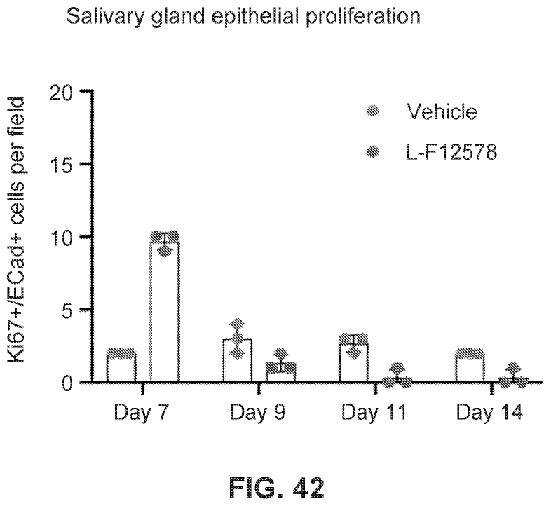

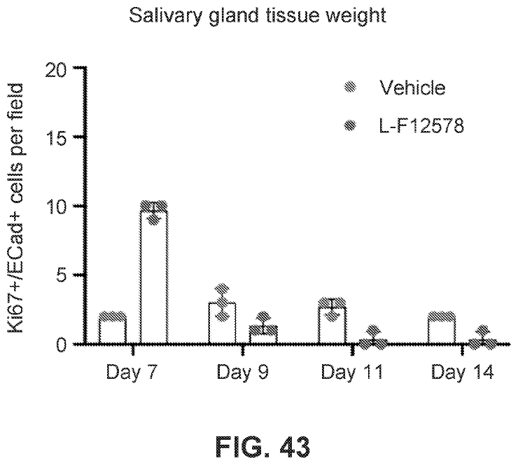

Sjögren's syndrome is a systemic autoimmune disease that particularly affects exocrine glands such as the salivary glands. Chronic inflammation in the glands with immune cell infiltration results in acinar cell atrophy leading to xerostomia (see, e.g., Jensen and Vissink (2014) Oral Maxillofac. Surg. Clin. North Am. 26:35-53). The MRL-lpr mouse strain, also known as lupus mice, exhibit systemic autoimmunity with similar salivary gland abnormalities in Sjögren's syndrome in humans such as reduced salivary production and lymphocyte infiltration (see, e.g., Ma et al. (2014) Diag. Pathol. 9:5). To test whether the L-F12578 WNT mimetic can increase and restore salivary gland weight in an ongoing damage environment, aged lupus and MRL/MpJ control mice were dosed for two weeks. Treatment with L-F12578 significantly increased salivary gland weight in both genetic strains without any observable adverse effect on histology (FIGS. 28A and 28B). Both the lupus mice and the control strain exhibited wet fur with mild color change upon treatment with L-F12578.

### X. Reagents and Materials

WNT mimetics were constructed as described in WO 2020/010308 A1 incorporated herein in its entirety herein. All recombinant proteins were produced in Expi293F™ cells (Thermo Fisher Scientific) by transient transfection unless otherwise specified. All IgG-based and Fc-containing constructs were first purified with Protein-A resin and eluted with 0.1 M glycine pH 3.5. All proteins were then polished by a size exclusion column in HBS buffer (10 mM HEPES pH 7.2, 150 mM NaCl). Proteins were supplemented with glycerol to 10% for long term storage at −80° C.

Reagents used in lacrimal gland regeneration experiments include (but are not limited to):

Reagent List

| Component | Catalog number |
|---|---|
| Advanced DMEM/F-12 | Thermo Scientific 12634-010 |
| HEPES | Thermo Scientific 15630080 |
| GlutaMAX | Thermo Scientific 35050061 |
| Penicillin-Streptomycin | Thermo Scientific 15140122 |
| B27 | Thermo Scientific 17504044 |
| N2 | Thermo Scientific 17502048 |
| Human Growth Factors | Peprotech |
| Recombinant Human R-Spondin 1 | R&D 4645RS |
| Surrogate WNT | In-house production |
| Matrigel ™ GFR Membrane Matrix | Corning CB40230C |
| Y-27632 | MilliporeSigma 5092280001 |

| Primer name | Primer sequence | SEQ ID No. |
|---|---|---|
| Mouse forward Axin2 | CAGCCCAAGAACCGGGAAAT | 17 |
| Mouse reverse Axin2 | GAGCCTCCTCTCTTTTACAGCA | 18 |
| Mouse forward Actb | ACCTTCTACAATGAGCTGCGT | 19 |
| Mouse reverse Actb | AGGTCTCAAACATGATCTGGGT | 20 |
| Mouse forward Rnf43 | CGGAGCCGTACTGGGTTAAG | 21 |
| Mouse reverse Rnf43 | GGCACGGGGATGAAACAAAT | 22 |
| Mouse forward Mist1 | CCAAGATCGAGACCCTCACG | 23 |
| Mouse reverse Mist1 | GCGGCTGCTGGACATAGTAA | 24 |
| Mouse forward Krt7 | GCGGAGATGAACCGCTCTAT | 25 |
| Mouse reverse Krt7 | TCTAACTTGGCACGCTGGTT | 26 |
| Human forward AXIN2 | TCATTTCCCGAGAACCCACC | 27 |
| Human reverse AXIN2 | AGCTTCAGCTTTTCCAGCCT | 28 |
| Human forward ACTB | CTGGAACGGTGAAGGTGACA | 29 |
| Human reverse ACTB | AAGGGACTTCCTGTAACAATGCA | 30 |
| Mouse forward Fzd1 | CATCGAGGCCAACTCACAGT | 31 |
| Mouse reverse Fzd1 | TGAGCCCCACAAAACACACT | 32 |
| Mouse forward Fzd2 | AGCGATTTCGCTACCCAGAG | 33 |
| Mouse reverse Fzd2 | AGAACGAAGCCCGCAATGTA | 34 |
| Mouse forward Fzd3 | GCCCTTTGTGAGACCAGGTT | 35 |
| Mouse reverse Fzd3 | ATATGCCTGCAGGTCTCAGC | 36 |
| Mouse forward Fzd4 | CCTGAGCGCCCCATCATATT | 37 |
| Mouse reverse Fzd4 | CTTTCCCGGCCTACAGTCAG | 38 |
| Mouse forward Fzd5 | CCGCCACAGGTACCTAGCTT | 39 |
| Mouse reverse Fzd5 | AAGGACAGAACTCTCGGAGGA | 40 |
| Mouse forward Fzd6 | CCCGAGGTGAGGCGTCC | 41 |
| Mouse reverse Fzd6 | TGAATTCTGGGGCAACTGCT | 42 |
| Mouse forward Fzd7 | ATGCTCTATGTACGCACCCG | 43 |
| Mouse reverse Fzd7 | CGAACTTGTTCATGAGCGCC | 44 |
| Mouse forward Fzd8 | TGTTGGAAGTGACCTCGCTC | 45 |
| Mouse reverse Fzd8 | AACCGATGCCTTTGCACAAC | 46 |
| Mouse forward Fzd9 | TAAGGACTTCGCGCTGGTTT | 47 |
| Mouse reverse Fzd9 | AAGATAATCGGGCGCTCTGG | 48 |
| Mouse forward Fzd10 | CCTTCCTCATCGACCCATCG | 49 |
| Mouse reverse Fzd10 | TAGCCCACCGAATAAACGCA | 50 |
| Mouse forward Lrp5 | CCATTGTGTTGCACCCTGTG | 51 |
| Mouse reverse Lrp5 | CAGGACATGCCGATCTCTCC | 52 |
| Mouse forward Lrp6 | TGCAAACAGACGGGACTTGA | 53 |
| Mouse reverse Lrp6 | CCAAACACAAAGTCCACCGC | 54 |

WNT mimetic constructs used in the experiments comprise the following amino acid sequences (Bold=LRP binding variable domain; underline=linker; italics=FZD binding variable domain; Plain (non-bold, non-underline, and non-italics=Fc domain).

| | |
|---|---|
| L-F12578 | Heavy Chain SEQ ID: 1 |
| (Fv-IgG) | EVQLVESGGGLVQPGGSLRLSCAASGFTFTSYYISWVRQAPGKGLEWVA |
| | EISPYSGSTYYADSVKGRFTISADTSKNTAYLQMNSLRAEDTAVYYCALR |
| | ARPPIRLHPRGSVMDYWGQGTLVTVSSGGGGSGGGGSGGGGSEVQLVESG |
| | GGLVQPGGSLRLSCAASGFTFSHYTLSWVRQAPGKGLEWVSVISGDGSYTYYADSV |
| | KGRFTISSDNSKNTLYLQMNSLRAEDTAVYYCARNFIKYVFANWGQGTLVTVSSAST |
| | KGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTC |
| | PPCPAPEAAGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALG |
| | APIEKTISKAKGQPREPQVYTLPPSREEMTKNQVSLTCLVKGFYPSDIAVEWES |
| | NGQPENNYKTTPPVLDSDGSFFLYSKLTVDKSRWQQGNVFSCSVMHEALHN |
| | HYTQKSLSLSPGK |
| | Light Chain SEQ ID: 2 |
| | DIQMTQSPSSLSASVGDRVTITCRASQDVSTAVAWYQQKPGKAPKLLIYS |
| | ASFLYSGVPSRFSGSGSGTDFTLTISSLQPEDFATYYCQQSYTTPPTFGQGT |
| | KVEIKGGGGSGGGGSGGGGSDIELTQPPSVSVAPGQTARISCSGDNIGSFYVHWY |
| | QQKPGQAPVLVIYDKSNRPSGIPERFSGSNSGNTATLTISGTQAEDEADYYCQSYAN |
| | TLSLVFGGGTKLTVLGQPKAAPSVTLFPPSSEELQANKATLVCLISDFYPGAVTV |
| | AWKADSSPVKAGVETTTPSKQSNNKYAASSYLSLTPEQWKSHRSYSCQVTHE |
| | GSTVEKTVAPTECS |
| L-F127 | Heavy Chain SEQ ID: 3 |
| (Fv-Ig) | EVQLVESGGGLVQPGGSLRLSCAASGFTFTSYYISWVRQAPGKGLEWVA |
| | EISPYSGSTYYADSVKGRFTISADTSKNTAYLQMNSLRAEDTAVYYCALR |
| | ARPPIRLHPRGSVMDYWGQGTLVTVSSGGGGSGGGGSGGGGSEVQLVESG |
| | GGLVQPGGSLRLSCAASGFNISSSYIHWVRQAPGKGLEWVAYIYSSYGSTYYADSVK |
| | GRFTISADTSKNTAYLQMNSLRAEDTAVYYCARASWYALDYWGQGTLVTVSSASTK |
| | GPSVFPLAPSSKSTSGGTAALGCLVKDYFPEPVTVSWNSGALTSGVHTFPAVL |
| | QSSGLYSLSSVVTVPSSSLGTQTYICNVNHKPSNTKVDKKVEPKSCDKTHTCP |
| | PCPAPEAAGGPSVFLFPPKPKDTLMISRTPEVTCVVVDVSHEDPEVKFNWYVD |
| | GVEVHNAKTKPREEQYNSTYRVVSVLTVLHQDWLNGKEYKCKVSNKALGA |
| | PIEKTISKAKGQPREPQVYTLPPSREEMTKNQVSLTCLVKGFYPSDIAVEWESN |
| | GQPENNYKTTPPVLDSDGSFFLYSKLTVDKSRWQQGNVFSCSVMHEALHNH |
| | YTQKSLSLSPGK |
| | Light Chain SEQ ID: 4 |
| | DIQMTQSPSSLSASVGDRVTITCRASQDVSTAVAWYQQKPGKAPKLLIYS |
| | ASFLYSGVPSRFSGSGSGTDFTLTISSLQPEDFATYYCQQSYTTPPTFGQGT |
| | KVEIKGGGGSGGGGSGGGGSDIQMTQSPSSLSASVGDRVTITCRASQSVSSAVAW |
| | YQQKPGKAPKLLIYSASSLYSGVPSRFSGSRSGTDFTLTISSLQPEDFATYYCQQYWY |
| | GVAPITFGQGTKVEIKRTVAAPSVFIFPPSDEQLKSGTASVVCLLNNFYPREAKV |
| | QWKVDNALQSGNSQESVTEQDSKDSTYSLSSTLTLSKADYEKHKVYACEVT |
| | HQGLSSPVTKSFNRGEC |
| L-F58 | Heavy Chain SEQ ID: 5 |
| (Fv-IgG) | EVQLVESGGGLVQPGGSLRLSCAASGFTFTSYYISWVRQAPGKGLEWVA |
| | EISPYSGSTYYADSVKGRFTISADTSKNTAYLQMNSLRAEDTAVYYCALR |
| | ARPPIRLHPRGSVMDYWGQGTLVTVSSGGGGSGGGGSGGGGSEVQLVESG |
| | GGLVQPGGSLRLSCAASGENISYSYIHWVRQAPGKGLEWVASIYSSSGSTSYADSVK |
| | GRFTISADTSKNTAYLQMNSLRAEDTAVYYCARGAIDYWGQGTLVTVSSASTKGPS |
| | VFPLAPSSKSTSGGTAALGCLVKDYFPEPVTVSWNSGALTSGVHTFPAVLQSS |
| | GLYSLSSVVTVPSSSLGTQTYICNVNHKPSNTKVDKKVEPKSCDKTHTCPPCP |
| | APEAAGGPSVFLFPPKPKDTLMISRTPEVTCVVVDVSHEDPEVKFNWYVDGV |
| | EVHNAKTKPREEQYNSTYRVVSVLTVLHQDWLNGKEYKCKVSNKALGAPIE |
| | KTISKAKGQPREPQVYTLPPSREEMTKNQVSLTCLVKGFYPSDIAVEWESNGQ |
| | PENNYKTTPPVLDSDGSFFLYSKLTVDKSRWQQGNVFSCSVMHEALHNHYT |
| | QKSLSLSPGK |
| | Light Chain SEQ ID: 6 |
| | DIQMTQSPSSLSASVGDRVTITCRASQDVSTAVAWYQQKPGKAPKLLIYS |
| | ASFLYSGVPSRFSGSGSGTDFTLTISSLQPEDFATYYCQQSYTTPPTFGQGT |
| | KVEIKGGGGSGGGGSGGGGSDIQMTQSPSSLSASVGDRVTITCRASQSVSSAVAW |
| | YQQKPGKAPKLLIYSASSLYSGVPSRFSGSRSGTDFTLTISSLQPEDFATYYCQQWYS |
| | SGHVLITFGQGTKVEIKRTVAAPSVFIFPPSDEQLKSGTASVVCLLNNFYPREAK |
| | VQWKVDNALQSGNSQESVTEQDSKDSTYSLSSTLTLSKADYEKHKVYACEV |
| | THQGLSSPVTKSFNRGEC |
| L-F4 | Heavy Chain SEQ ID: 7 |
| (Fv-IgG) | EVQLVESGGGLVQPGGSLRLSCAASGFTFTSYYISWVRQAPGKGLEWVA |
| | EISPYSGSTYYADSVKGRFTISADTSKNTAYLQMNSLRAEDTAVYYCALR |
| | ARPPIRLHPRGSVMDYWGQGTLVTVSSGGGGSGGGGSGGGGSEVQLVESG |
| | GGLVQPGGSLRLSCAASGFNISYYYIHWVRQAPGKGLEWVASIYPSSGYTYYADSVK |
| | GRFTISADTSKNTAYLQMNSLRAEDTAVYYCARSSFYWAMDYWGQGTLVTVSSAST |
| | KGPSVFPLAPSSKSTSGGTAALGCLVKDYFPEPVTVSWNSGALTSGVHTFPAV |
| | LQSSGLYSLSSVVTVPSSSLGTQTYICNVNHKPSNTKVDKKVEPKSCDKTHTC |
| | PPCPAPEAAGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNKALG |
| | APIEKTISKAKGQPREPQVYTLPPSREEMTKNQVSLTCLVKGFYPSDIAVEWES |
| | NGQPENNYKTTPPVLDSDGSFFLYSKLTVDKSRWQQGNVFSCSVMHEALHN |
| | HYTQKSLSLSPGK |
| | Light Chain SEQ ID: 8 |
| | DIQMTQSPSSLSASVGDRVTITCRASQDVSTAVAWYQQKPGKAPKLLIYS |
| | ASFLYSGVPSRFSGSGSGTDFTLTISSLQPEDFATYYCQQSYTTPPTFGQGT |
| | KVEIKGGGGSGGGGSGGGGSDIQMTQSPSSLSASVGDRVTITCRASQSVSSAVAW |
| | YQQKPGKAPKLLIYSASSLYSGVPSRFSGSRSGTDFTLTISSLQPEDFATYYCQQSYA |
| | AYLFTFGQGTKVEIKRTVAAPSVFIFPPSDEQLKSGTASVVCLLNNFYPREAKV |
| | QWKVDNALQSGNSQESVTEQDSKDSTYSLSSTLTLSKADYEKHKVYACEVT |
| | HQGLSSPVTKSFNRGEC |
| L-F10 | Heavy Chain SEQ ID: 9 |
| (Fv-IgG) | EVQLVESGGGLVQPGGSLRLSCAASGFTFTSYYISWVRQAPGKGLEWVA |
| | EISPYSGSTYYADSVKGRFTISADTSKNTAYLQMNSLRAEDTAVYYCALR |
| | ARPPIRLHPRGSVMDYWGQGTLVTVSSGGGGSGGGGSGGGGSEVQLVESG |
| | GGLVQPGGSLRLSCAASGFTFSSFNMFWVRQAPGKGLEWVAGIDDDGSYPNYGS |
| | AVKGRATISRDNSKNTLYLQMNSLRAEDTAVYYCAKSGYGGSWGGYIADDIDAWG |
| | QGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKV |
| | EPKSCDKTHTCPPCPAPEAAGGPSVFLFPPKPKDTLMISRTPEVTCVVVDVSHE |
| | DPEVKFNWYVDGVEVHNAKTKPREEQYNSTYRVVSVLTVLHQDWLNGKEY |
| | KCKVSNKALGAPIEKTISKAKGQPREPQVYTLPPSREEMTKNQVSLTCLVKGF |
| | YPSDIAVEWESNGQPENNYKTTPPVLDSDGSFFLYSKLTVDKSRWQQGNVFS |
| | CSVMHEALHNHYTQKSLSLSPGK |
| | Light Chain SEQ ID: 10 |
| | DIQMTQSPSSLSASVGDRVTITCRASQDVSTAVAWYQQKPGKAPKLLIYS |
| | ASFLYSGVPSRFSGSGSGTDFTLTISSLQPEDFATYYCQQSYTTPPTFGQGT |
| | KVEIKGGGGSGGGGSGGGGSELTQPPSVSVSPGQTARITCSGDGSYAGSYYYGW |
| | YQQKPGQAPVTLIYYNNKRPSGIPERFSGSLSGSTNTLTISGVQAEDEADYYCGSAD |
| | NSGAAFGGGTKLTVLRTVAAPSVFIFPPSDEQLKSGTASVVCLLNNFYPREAKV |
| | QWKVDNALQSGNSQESVTEQDSKDSTYSLSSTLTLSKADYEKHKVYACEVT |
| | HQGLSSPVTKSFNRGEC |
| 1SH1-03 | Light Chain SEQ ID: 11 |
| | DVQLVESGGGLVQPGGSLRLSCTSSANINSIETLGWYRQAPGKQRELIAN |
| | MRGGGYMKYAGSLKGRFTMSTESAKNTMYLQMNSLKPEDTAVYYCYV |
| | KLRDDDYVYRGQGTQVTVSSGGSGSDVVMTQSPLSLPVTPGEPASISCRSSQSL |
| | LHSNGYNYLDWYLQKPGQSPQLLIYLGSKRASGVPDRFSGSGSGTDFTLKISRVEA |
| | EDVGVYYCMQALQIPPTFGQGTKVEIKRTVAAPSVFIFPPSDEQLKSGTASVVCL |
| | LNNFYPREAKVQWKVDNALQSGNSQESVTEQDSKDSTYSLSSTLTLSKADYE |
| | KHKVYACEVTHQGLSSPVTKSFNRGEC |
| 1SH1-26 | Light Chain SEQ ID: 12 |
| | DVQLVESGGGLVQAGGSLRLACAGSGRIFAIYDIAWYRHPPGNQRELVA |
| | MIRPVVTEIDYADSVKGRFTISRNNAMKTVYLQMNNLKPEDTAVYYCNA |
| | KRPWGSRDEYWGQGTQVTVSSGGSGSDVVMTQSPLSLPVTPGEPASISCRSSQ |
| | SLLHSNGYNYLDWYLQKPGQSPQLLIYLGSKRASGVPDRFSGSGSGTDFTLKISRVE |
| | AEDVGVYYCMQALQIPPTFGQGTKVEIKRTVAAPSVFIFPPSDEQLKSGTASVVC |
| | LLNNFYPREAKVQWKVDNALQSGNSQESVTEQDSKDSTYSLSSTLTLSKADY |
| | EKHKVYACEVTHQGLSSPVTKSFNRGEC |
| 1SH1-36 | Light Chain SEQ ID: 13 |
| | QVKLEESGGGLVQAGGSLRLSCAASGRIFSIYDMGWFRQAPGKEREFVS |
| | GIRWSGGTSYADSVKGRFTISKDNAKNTIYLQMNNLKAEDTAVYYCGSR |
| | GYWGQGTLVTVSSGGSGSDVVMTQSPLSLPVTPGEPASISCRSSQSLLHSNGYN |
| | YLDWYLQKPGQSPQLLIYLGSKRASGVPDRFSGSGSGTDFTLKISRVEAEDVGVYY |
| | CMQALQIPPTFGQGTKVEIKRTVAAPSVFIFPPSDEQLKSGTASVVCLLNNFYPR |
| | EAKVQWKVDNALQSGNSQESVTEQDSKDSTYSLSSTLTLSKADYEKHKVYA |
| | CEVTHQGLSSPVTKSFNRGEC |
| 1SH1-03 | Heavy Chain SEQ ID: 14 |
| 1SH1-26 | EVQLVESGPGLVKPSQTLSLTCTVSGGSISSGGYYWSWIRQHPGKGLEWIGYIYYSG |
| 1SH1-36 | STYYNPSLKSRVTISVDTSKNQFSLKLNSVTAADTAVYYCARHAGFYGLADYFDYWG |
| | QGTLVTVSSASTKGPSVFPLAPSSKSTSGGTAALGCLVKDYFPEPVTVSWNSGA |
| | LTSGVHTFPAVLQSSGLYSLSSVVTVPSSSLGTQTYICNVNHKPSNTKVDKKV |
| | EPKSCDKTHTCPPCPAPEAAGGPSVFLFPPKPKDTLMISRTPEVTCVVVDVSHE |
| | DPEVKFNWYVDGVEVHNAKTKPREEQYNSTYRVVSVLTVLHQDWLNGKEY |
| | KCKVSNKALGAPIEKTISKAKGQPREPQVYTLPPSREEMTKNQVSLTCLVKGF |
| | YPSDIAVEWESNGQPENNYKTTPPVLDSDGSFFLYSKLTVDKSRWQQGNVFS |
| | CSVMHEALHNHYTQKSLSLSPGK |
| L-F49 | Light Chain SEQ ID: 15 |
| | DIQMTQSPSSLSASVGDRVTITCRASQDVSTAVAWYQQKPGKAPKLLIYS |
| | ASFLYSGVPSRFSGSGSGTDFTLTISSLQPEDFATYYCQQSYTTPPTFGQGT |
| | KVEIKRGGGGSGGGGSGGGGSDIQMTQSPSSPSASVGDRVTITCRASQSISSYL |
| | NWYQQKPGKAPKLLIYAASSLQSGVPSRFSGSGSGTDFTLTISSLQPEDFATYY |
| | CQQSYSTPLTFGGGTKVEIKRTVAAPSVFIFPPSDEQLKSGTASVVCLLNNFYP |
| | REAKVQWKVDNALQSGNSQESVTEQDSKDSTYSLSSTLTLSKADYEKHKVY |
| | ACEVTHQGLSSPVTKSFNRGEC |
| | Heavy Chain SEQ ID: 16 |
| | EVQLVESGGGLVQPGGSLRLSCAASGFTFTSYYISWVRQAPGKGLEWVA |
| | EISPYSGSTYYADSVKGRFTISADTSKNTAYLQMNSLRAEDTAVYYCALR |
| | ARPPIRLHPRGSVMDYWGQGTLVTVSSGGGGSGGGGSGGGGSEVQLVQS |
| | GAEVKKPGASVKVSCKASGGTFSSYAISWVRQAPGQGLEWMGWINAGN |
| | GNTTYAQKFQGRVTMTRDTSTSTVYMELSSLRSEDTAVYYCARHYYGSG |
| | SYPDWGQGTLVTVSSASTKGPSVFPLAPSSKSTSGGTAALGCLVKDYFPEPV |
| | TVSWNSGALTSGVHTFPAVLQSSGLYSLSSVVTVPSSSLGTQTYICNVNHKPS |
| | NTKVDKKVEPKSCDKTHTCPPCPAPEAAGGPSVFLFPPKPKDTLMISRTPEVT |
| | CVVVDVSHEDPEVKFNWYVDGVEVHNAKTKPREEQYNSTYRVVSVLTVLH |
| | QDWLNGKEYKCKVSNKALGAPIEKTISKAKGQPREPQVYTLPPSREEMTKNQ |
| | VSLTCLVKGFYPSDIAVEWESNGQPENNYKTTPPVLDSDGSFFLYSKLTVDKS |
| | RWQQGNVFSCSVMHEALHNHYTQKSLSLSPGK |

It will be readily understood to those skilled in the art that the above constructs may be altered and expressed as various homologs and isoforms, may be edited at non-binding-domain sequences, and may be expressed using various synonymous nucleotide sequences using various suitable expression vector systems.

### REFERENCES

The following references are incorporated herein in their entireties:
Marie Bannier-Hélaouët et al., Exploring the human lacrimal gland using organoids and single-cell sequencing, CELL STEM CELL (2021);
Liana. Basova et at, Origin and Lineage Plasticity of Endogenous Lacrimal Gland Epithelial Stem Progenitor Cells, ISCIENCE 23.6 (2020): 101230;
Darlene A. Dartt, Signal transduction and control of lacrimal gland protein secretion: A review, 8 CURRENT EYE RES. 619 (1989);
Charlotte H. Dean et al., Canonical Wnt signaling negatively regulates branching morphogenesis of the lung and lacrimal gland, 286 DEVELOPMENTAL BIO. 270 (2005);
T Farmer D'Juan et al., Defining epithelial cell dynamics and lineage relationships in the developing lacrimal gland, DEVELOPMENT 144.13 (2017): 2517-28;
Galina Dvoriantchikova et al., Molecular Profiling of the Developing Lacrimal Gland Reveals Putative Role of Notch Signaling in Branching Morphogenesis, 58 INVEST. OPHTHALMOL. VIS. SCI. 1098 (2017);
Ankur Garg & Xin Zhang, Lacrimal Gland Development: From Signaling Interactions to Regenerative Medicine, 246 DEVELOPMENTAL DYNAMICS 970 (2017);
Hodges, Robin R., and Darlene A. Dartt. “Regulatory pathways in lacrimal gland epithelium.” International review of cytology 231 (2003): 129-196;
John A. MacMillan, Diseases of the Lacrimal Gland and Ocular Complications, 138 JAMA 801 (1948); Shubha Tiwari, Human lacrimal gland regeneration: Perspective and review of literature, 28 SAUDI J. OPHTHALMOL. 12 (2014);
Yupeng Yao & Yan Zhang, The lacrimal gland: development, wound repair and regeneration, 39 BIOTECH. LETTERS 939 (2017);
Driss Zoukhri et al. A Single Injection of Interleukin-1 Induces a Reversible Aqueous-tear Deficiency, Lacrimal Gland Inflammation, and Acinar and Ductal Cell Prolferation, 84 EXP. EYE RES. 894-904 (2007);
Darlene A. Dartt, OCULAR DISEASE: MECHANISMS AND MANAGEMENT 105-13 (Leonard A. Levin & Daniel M. Albert eds., 2010);
Liu, Y., Hirayama, M., Kawakita, T. and Tsubota, K., 2017. A ligation of the lacrimal excretory duct in mouse induces lacrimal gland inflammation with prolferative cells. Stem Cells International, 2017.;
Zhang, Z., Broderick, C., Nishimoto, M., Yamaguchi, T., Lee, S. J., Zhang, H., Chen, H., Patel, M., Ye, J., Ponce, A., et al. (2020). Tissue-targeted R-spondin mimetics for liver regeneration. Sci Rep 10, 13951;
Xie, Y., Zamponi, R., Charlat, O., Ramones, M., Swalley, S., Jiang, X., Rivera, D., Tschantz, W., Lu, B., Quinn, L., et al. (2013). Interaction with both ZNRF3 and LGR4 is required for the signalling activity of R-spondin. EMBO Rep 14, 1120-1126. 10.1038/embor.2013.167.

1. A method of regenerating lacrimal gland cells in a subject, comprising administering a WNT signaling modulator to the subject.

2. The method of claim 1 wherein the lacrimal gland cells are acinar cells, progenitor cells, ductal cells, myoepithelial cells, or immune cells.

3. The method of claim 1, wherein the WNT signaling modulator is an engineered WNT signaling modulator.

4. The method of claim 3, wherein the engineered WNT signaling modulator is a WNT agonist.

5. The method of claim 1, wherein the WNT signaling modulator is an engineered WNT superagonist comprising an E3 Ligase binding domain selected from the group consisting of: a mutant R-spondin (RSPO) protein; and an antibody specific for E3 ligase or functional fragment thereof.

6. The method of claim 1, wherein the WNT signaling modulator comprises at least one engineered bi-specific IgG antibody or antigen binding fragment thereof that directly activates a canonical WNT signaling pathway.

7. The method of claim 6, wherein the bi-specific IgG antibody or antigen binding fragment thereof comprises a binding composition specific for at least one FZD receptor and a binding composition specific for at least one LRP receptor.

8. The method of claim 1, wherein the WNT signaling modulator is selected from: (i) WNT3a; (ii) a WNT mimetic; or (iii) an R-spondin mimetic;
a) the FZD binding composition binds to at least one of Fzd1, Fzd2, Fzd5, Fzd7, Fzd8; and
b) the LRP binding composition binds to at least one of Lrp6, and/or Lrp5.

9. The method of claim 1, wherein the WNT signaling modulator binds to the group consisting of: Fzd5 and Fzd8, and Lrp6 and/or Lrp5.

10. The method of claim 1, wherein the WNT signaling modulator comprises one or more polypeptides selected from the group consisting of: SEQ ID NO: 1, SEQ ID NO: 2, SEQ ID: NO 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO:15, SEQ ID NO:16, and any isoform or homolog thereof.

11. The method of claim 10 wherein the WNT signaling modulator comprises:
a) two polypeptides of SEQ ID NO:1 and two polypeptides of SEQ ID NO:2, or variants thereof having at least 90% identity thereto;
b) two polypeptides of SEQ ID NO:3 and two polypeptides of SEQ ID NO:4, or variants thereof having at least 90% identity thereto;
c) two polypeptides of SEQ ID NO:5 and two polypeptides of SEQ ID NO:6, or variants thereof having at least 90% identity thereto;
d) two polypeptides of SEQ ID NO:7 and two polypeptides of SEQ ID NO:8, or variants thereof having at least 90% identity thereto; or
e) two polypeptides of SEQ ID NO:9 and two polypeptides of SEQ ID NO:10, or variants thereof having at least 90% identity thereto;
f) two polypeptides of SEQ ID NO:11 and two polypeptides of SEQ ID NO:14, or variants thereof having at least 90% identity thereof;
g) two polypeptides of SEQ ID NO:12 and two polypeptides of SEQ ID NO:14, or variants thereof having at least 90% identity thereof;
h) two polypeptides of SEQ ID NO:13 and two polypeptides of SEQ ID NO:14, or variants thereof having at least 90% identity thereof; or
i) two polypeptides of SEQ ID NO:15 and two polypeptides of SEQ ID NO:16, or variants thereof having at least 90% identity thereof.

12. The method of any of claims 1-11 wherein the method further comprises co-administration of at least one molecule from the group consisting of: RSPO2, RSPO2 fragment, and engineered RSPO2 mimetic.

13. The method of any of claims 1-12 wherein the subject is a mammal.

14. The method of any of claims 1-13 wherein the subject is a human.

15. The method of any of claims 1-14 wherein the subject is in need of lacrimal cell regeneration because of a lacrimal gland disorder.

16. The method of claim 15, wherein the lacrimal gland disorder is a dry eye disease.

17. The method of claim 16, wherein the dry eye disease is caused by Sjögren's syndrome, chronic graft versus host disease (cGHVD), rheumatoid arthritis (RA), Stephen's Johnson syndrome, ocular rosacea, chemotherapy, radiation oncology treatments, diabetes, lupus, or a Meibomian Gland Disorder (MGD).

18. The method of claim 17, wherein the dry eye disease is Sjögren's syndrome.

19. A method of treating a lacrimal gland disorder in a subject, comprising administering a WNT signaling modulator to the subject.

20. The method of claim 19, wherein the WNT signaling modulator is an engineered WNT signaling modulator.

21. The method of claim 19, wherein the WNT signaling modulator is an engineered WNT agonist.

22. The method of claim 19, wherein the WNT signaling modulator comprises at least one engineered bi-specific antibody or antigen binding fragment thereof that directly activates a canonical WNT signaling pathway.

23. The method of claim 19, wherein the engineered WNT agonist is selected from: (i) WNT3a; (ii) a WNT mimetic; or (iii) an R-spondin mimetic.

24. The method of claim 19, wherein the WNT signaling modulator binds to:
a) any one or more of the group consisting of: Fzd1, Fzd2, Fzd5, Fzd7, Fzd8; and
b) Lrp6, and/or Lrp5.

25. The method of claim 19, wherein the WNT signaling modulator binds to:
a) any one or more of the group consisting of: Fzd1, Fzd2, and Fzd7; and
b) Lrp6, and/or Lrp5.

26. The method of claim 20, wherein the WNT signaling modulator binds to:
a) any one or more of the group consisting of: Fzd5 and Fzd8; and
b) Lrp6 and/or, Lrp5.

27. The method of claim 19, wherein the WNT signaling modulator comprises a sequence selected from the group consisting of: SEQ ID NO: 1, SEQ ID NO: 2, SEQ ID: NO 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, and any isoform or homolog thereof of any of the foregoing, optionally wherein the WNT signaling modulator comprises:
a) two polypeptides of SEQ ID NO:1 (or variants of one or both having at least 90% identity thereto) and two polypeptides of SEQ ID NO:2 (or variants of one or both having at least 90% identity thereto);
b) two polypeptides of SEQ ID NO:3 (or variants of one or both having at least 90% identity thereto) and two polypeptides of SEQ ID NO:4 (or variants of one or both having at least 90% identity thereto);
c) two polypeptides of SEQ ID NO:5 (or variants of one or both having at least 90% identity thereto) and two polypeptides of SEQ ID NO:6 (or variants of one or both having at least 90% at least identity thereto);
d) two polypeptides of SEQ ID NO:7 (or variants of one or both having at least 90% identity thereto) and two polypeptides of SEQ ID NO:8 (or variants of one or both having at least 90% identity thereto); or
e) two polypeptides of SEQ ID NO:9 (or variants of one or both having at least 90% identity thereto) and two polypeptides of SEQ ID NO:10 (or variants of one or both having at least 90% identity thereto);
f) two polypeptides of SEQ ID NO:11 (or variants of one or both having at least 90%, at least 95%, or at least 98% identity thereto) and two polypeptides of SEQ ID NO:14 (or variants of one or both having at least 90% identity thereto);
g) two polypeptides of SEQ ID NO:12 (or variants of one or both having at least 90% identity thereto) and two polypeptides of SEQ ID NO:14 (or variants of one or both having at least 90%, identity thereto);
h) two polypeptides of SEQ ID NO:13 (or variants of one or both having at least 90%, identity thereto) and two polypeptides of SEQ ID NO:14 (or variants of one or both having at least 90%, identity thereto); or
i) two polypeptides of SEQ ID NO:15 (or variants of one or both having at least 90%, identity thereto) and two polypeptides of SEQ ID NO:16 (or variants of one or both having at least 90%, identity thereto).

28. The method of any of claims 19-27 wherein the method further comprises administering at least one of the group consisting of: RSPO2, an RSPO2 fragment, and an engineered RSPO2 mimetic.

29. The method of any of claims 19-28 wherein the WNT signaling modulator is administered in a therapeutically effective amount.

30. The method of any of claims 19-29 wherein the subject is a live mammal.

31. The method of any of claims 19-30 wherein the subject is a human patient.

32. The method of any of claims 19-31 wherein the lacrimal gland disorder is dry eye disease.

33. The method of claim 32, wherein the dry eye disease is caused by Sjögren's syndrome, chronic graft versus host disease (cGHVD), rheumatoid arthritis (RA), Stephen's Johnson syndrome, ocular rosacea, chemotherapy, radiation oncology treatments, diabetes, lupus, or a Meibomian Gland Disorder (MGD).

34. The method of claim 33, wherein the dry eye disease is caused by Sjögren's syndrome.

35. A composition for the treatment of a dry-eye disorder in a subject, the composition comprising a WNT signaling modulator.

36. The composition of claim 35, wherein the WNT signaling modulator comprises at least one engineered bi-specific antibody or antigen binding fragment thereof that directly activates a canonical WNT signaling pathway.

37. The composition of claim 35, wherein the at least one engineered bi-specific full-length IgG antibody or antigen binding fragment thereof is specific for or preferentially binds any one of the following combination of Fzds:
a) Fzd1, Fzd2, Fzd5, Fzd7, Fzd8;
b) Fzd 1, Fzd 2, and Fzd 7; or
c) Fzd5 and Fzd8,
wherein the engineered bi-specific antibody or antigen binding fragment thereof also binds Lrp 5 and/or Lrp6.

38. The composition of claim 35, further comprising an anti-inflammatory agent.

39. The composition of claims 35-38, wherein the composition comprises therapeutically effective amounts of its components.

40. The composition of claims 35-38, wherein the subject is a live mammal.

41. The composition of claims 35-38, wherein the subject is human patient.

42. The composition of claims 35-38, wherein the aqueous-deficient dry-eye disorder is caused by a Sjögren's syndrome, disorder, chronic graft versus host disease (cGHVD), rheumatoid arthritis (RA), Stephen's Johnson syndrome, ocular rosacea, chemotherapy, radiation oncology treatments, diabetes, lupus, or a Meibomian Gland Disorder (MGD.


SEQUENCE LISTING (54 sequences)

SEQ ID NO: 1 - protein, 589 aa, synthetic construct
  REGION 1..589: note = L6-F12578 (Fv-IgG) heavy chain
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 335 aa, synthetic construct
  REGION 1..335: note = L6-F12578 (Fv-IgG) Light Chain
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 3 - protein, 588 aa, synthetic construct
  REGION 1..588: note = L6-F127 (Fv-Ig) heavy chain
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFNISSSYIHWVRQAPGKGLEWVAYIYSSYGSTYYADSVKGRFTISADTSKNTAYLQMNSLRAEDTAVYYCARASWYALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 338 aa, synthetic construct
  REGION 1..338: note = L6-F127 (Fv-Ig) light chain
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQYWYGVAPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 585 aa, synthetic construct
  REGION 1..585: note = L6-F58 (Fv-IgG) heavy chain
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFNISYSYIHWVRQAPGKGLEWVASIYSSSGSTSYADSVKGRFTISADTSKNTAYLQMNSLRAEDTAVYYCARGAIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 339 aa, synthetic construct
  REGION 1..339: note = L6-F58 (Fv-IgG) light chain
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQWYSSGHVLITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 589 aa, synthetic construct
  REGION 1..589: note = L6-F4 (Fv-IgG) heavy chain
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFNISYYYIHWVRQAPGKGLEWVASIYPSSGYTYYADSVKGRFTISADTSKNTAYLQMNSLRAEDTAVYYCARSSFYWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 337 aa, synthetic construct
  REGION 1..337: note = L6-F4 (Fv-IgG) light chain
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSDIQMTQSPSSLSASVGDRVTITCRASQSVSSAVAWYQQKPGKAPKLLIYSASSLYSGVPSRFSGSRSGTDFTLTISSLQPEDFATYYCQQSYAAYLFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 597 aa, synthetic construct
  REGION 1..597: note = L6-F10 (Fv-IgG) heavy chain
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVESGGGLVQPGGSLRLSCAASGFTFSSFNMFWVRQAPGKGLEWVAGIDDDGSYPNYGSAVKGRATISRDNSKNTLYLQMNSLRAEDTAVYYCAKSGYGGSWGGYIADDIDAWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 335 aa, synthetic construct
  REGION 1..335: note = L6-F10 (Fv-IgG) light chain
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKGGGGSGGGGSGGGGSELTQPPSVSVSPGQTARITCSGDGSYAGSYYYGWYQQKPGQAPVTLIYYNNKRPSGIPERFSGSLSGSTNTLTISGVQAEDEADYYCGSADNSGAAFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 341 aa, synthetic construct
  REGION 1..341: note = 1SH1-03 light chain
DVQLVESGGGLVQPGGSLRLSCTSSANINSIETLGWYRQAPGKQRELIANMRGGGYMKYAGSLKGRFTMSTESAKNTMYLQMNSLKPEDTAVYYCYVKLRDDDYVYRGQGTQVTVSSGGSGSDVVMTQSPLSLPVTPGEPASISCRSSQSLLHSNGYNYLDWYLQKPGQSPQLLIYLGSKRASGVPDRFSGSGSGTDFTLKISRVEAEDVGVYYCMQALQIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 343 aa, synthetic construct
  REGION 1..343: note = 1SH1-26 light chain
DVQLVESGGGLVQAGGSLRLACAGSGRIFAIYDIAWYRHPPGNQRELVAMIRPVVTEIDYADSVKGRFTISRNNAMKTVYLQMNNLKPEDTAVYYCNAKRPWGSRDEYWGQGTQVTVSSGGSGSDVVMTQSPLSLPVTPGEPASISCRSSQSLLHSNGYNYLDWYLQKPGQSPQLLIYLGSKRASGVPDRFSGSGSGTDFTLKISRVEAEDVGVYYCMQALQIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 335 aa, synthetic construct
  REGION 1..335: note = 1SH1-36 light chain
QVKLEESGGGLVQAGGSLRLSCAASGRIFSIYDMGWFRQAPGKEREFVSGIRWSGGTSYADSVKGRFTISKDNAKNTIYLQMNNLKAEDTAVYYCGSRGYWGQGTLVTVSSGGSGSDVVMTQSPLSLPVTPGEPASISCRSSQSLLHSNGYNYLDWYLQKPGQSPQLLIYLGSKRASGVPDRFSGSGSGTDFTLKISRVEAEDVGVYYCMQALQIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 453 aa, synthetic construct
  REGION 1..453: note = 1SH1-03 or 1SH1-26 or 1SH1-36 heavy chain
EVQLVESGPGLVKPSQTLSLTCTVSGGSISSGGYYWSWIRQHPGKGLEWIGYIYYSGSTYYNPSLKSRVTISVDTSKNQFSLKLNSVTAADTAVYYCARHAGFYGLADYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 337 aa, synthetic construct
  REGION 1..337: note = L-F49 Light Chain
DIQMTQSPSSLSASVGDRVTITCRASQDVSTAVAWYQQKPGKAPKLLIYSASFLYSGVPSRFSGSGSGTDFTLTISSLQPEDFATYYCQQSYTTPPTFGQGTKVEIKRGGGGSGGGGSGGGGSDIQMTQSPSSPSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 590 aa, synthetic construct
  REGION 1..590: note = L-F49 Heavy Chain
EVQLVESGGGLVQPGGSLRLSCAASGFTFTSYYISWVRQAPGKGLEWVAEISPYSGSTYYADSVKGRFTISADTSKNTAYLQMNSLRAEDTAVYYCALRARPPIRLHPRGSVMDYWGQGTLVTVSSGGGGSGGGGSGGGGSEVQLVQSGAEVKKPGASVKVSCKASGGTFSSYAISWVRQAPGQGLEWMGWINAGNGNTTYAQKFQGRVTMTRDTSTSTVYMELSSLRSEDTAVYYCARHYYGSGSYPDWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 20 bp, synthetic construct
  source 1..20: mol_type = other DNA
cagcccaagaaccgggaaat

SEQ ID NO: 18 - DNA, 22 bp, synthetic construct
  source 1..22: mol_type = other DNA
gagcctcctctcttttacagca

SEQ ID NO: 19 - DNA, 21 bp, synthetic construct
  source 1..21: mol_type = other DNA
accttctacaatgagctgcgt

SEQ ID NO: 20 - DNA, 22 bp, synthetic construct
  source 1..22: mol_type = other DNA
aggtctcaaacatgatctgggt

SEQ ID NO: 21 - DNA, 20 bp, synthetic construct
  source 1..20: mol_type = other DNA
cggagccgtactgggttaag

SEQ ID NO: 22 - DNA, 20 bp, synthetic construct
  source 1..20: mol_type = other DNA
ggcacggggatgaaacaaat

SEQ ID NO: 23 - DNA, 20 bp, synthetic construct
  source 1..20: mol_type = other DNA
ccaagatcgagaccctcacg

SEQ ID NO: 24 - DNA, 20 bp, synthetic construct
  source 1..20: mol_type = other DNA
gcggctgctggacatagtaa

SEQ ID NO: 25 - DNA, 20 bp, synthetic construct
  source 1..20: mol_type = other DNA
gcggagatgaaccgctctat

SEQ ID NO: 26 - DNA, 20 bp, synthetic construct
  source 1..20: mol_type = other DNA
tctaacttggcacgctggtt

SEQ ID NO: 27 - DNA, 20 bp, synthetic construct
  source 1..20: mol_type = other DNA
tcatttcccgagaacccacc

SEQ ID NO: 28 - DNA, 20 bp, synthetic construct
  source 1..20: mol_type = other DNA
agcttcagcttttccagcct

SEQ ID NO: 29 - DNA, 20 bp, synthetic construct
  source 1..20: mol_type = other DNA
ctggaacggtgaaggtgaca

SEQ ID NO: 30 - DNA, 23 bp, synthetic construct
  source 1..23: mol_type = other DNA
aagggacttcctgtaacaatgca

SEQ ID NO: 31 - DNA, 20 bp, synthetic construct
  source 1..20: mol_type = other DNA
catcgaggccaactcacagt

SEQ ID NO: 32 - DNA, 20 bp, synthetic construct
  source 1..20: mol_type = other DNA
tgagccccacaaaacacact

SEQ ID NO: 33 - DNA, 20 bp, synthetic construct
  source 1..20: mol_type = other DNA
agcgatttcgctacccagag

SEQ ID NO: 34 - DNA, 20 bp, synthetic construct
  source 1..20: mol_type = other DNA
agaacgaagcccgcaatgta

SEQ ID NO: 35 - DNA, 20 bp, synthetic construct
  source 1..20: mol_type = other DNA
gccctttgtgagaccaggtt

SEQ ID NO: 36 - DNA, 20 bp, synthetic construct
  source 1..20: mol_type = other DNA
atatgcctgcaggtctcagc

SEQ ID NO: 37 - DNA, 20 bp, synthetic construct
  source 1..20: mol_type = other DNA
cctgagcgccccatcatatt

SEQ ID NO: 38 - DNA, 20 bp, synthetic construct
  source 1..20: mol_type = other DNA
ctttcccggcctacagtcag

SEQ ID NO: 39 - DNA, 20 bp, synthetic construct
  source 1..20: mol_type = other DNA
ccgccacaggtacctagctt

SEQ ID NO: 40 - DNA, 21 bp, synthetic construct
  source 1..21: mol_type = other DNA
aaggacagaactctcggagga

SEQ ID NO: 41 - DNA, 17 bp, synthetic construct
  source 1..17: mol_type = other DNA
cccgaggtgaggcgtcc

SEQ ID NO: 42 - DNA, 20 bp, synthetic construct
  source 1..20: mol_type = other DNA
tgaattctggggcaactgct

SEQ ID NO: 43 - DNA, 20 bp, synthetic construct
  source 1..20: mol_type = other DNA
atgctctatgtacgcacccg

SEQ ID NO: 44 - DNA, 20 bp, synthetic construct
  source 1..20: mol_type = other DNA
cgaacttgttcatgagcgcc

SEQ ID NO: 45 - DNA, 20 bp, synthetic construct
  source 1..20: mol_type = other DNA
tgttggaagtgacctcgctc

SEQ ID NO: 46 - DNA, 20 bp, synthetic construct
  source 1..20: mol_type = other DNA
aaccgatgcctttgcacaac

SEQ ID NO: 47 - DNA, 20 bp, synthetic construct
  source 1..20: mol_type = other DNA
taaggacttcgcgctggttt

SEQ ID NO: 48 - DNA, 20 bp, synthetic construct
  source 1..20: mol_type = other DNA
aagataatcgggcgctctgg

SEQ ID NO: 49 - DNA, 20 bp, synthetic construct
  source 1..20: mol_type = other DNA
ccttcctcatcgacccatcg

SEQ ID NO: 50 - DNA, 20 bp, synthetic construct
  source 1..20: mol_type = other DNA
tagcccaccgaataaacgca

SEQ ID NO: 51 - DNA, 20 bp, synthetic construct
  source 1..20: mol_type = other DNA
ccattgtgttgcaccctgtg

SEQ ID NO: 52 - DNA, 20 bp, synthetic construct
  source 1..20: mol_type = other DNA
caggacatgccgatctctcc

SEQ ID NO: 53 - DNA, 20 bp, synthetic construct
  source 1..20: mol_type = other DNA
tgcaaacagacgggacttga

SEQ ID NO: 54 - DNA, 20 bp, synthetic construct
  source 1..20: mol_type = other DNA
ccaaacacaaagtccaccgc